SEQUENCE LISTING (44 sequences)

SEQ ID NO: 1 - protein, 16 aa, Artificial Sequence
  Description of Artificial Sequence Synthetic peptide
CARRYFGVVADAFDIW

SEQ ID NO: 2 - protein, 16 aa, Artificial Sequence
  Description of Artificial Sequence Synthetic peptide
CARPKSDYIIDAFDIW

SEQ ID NO: 3 - protein, 14 aa, Artificial Sequence [sequence omitted]

SEQ ID NO: 4 - protein, 5 aa, Artificial Sequence
  Description of Artificial Sequence Synthetic peptide
DYIID

SEQ ID NO: 5 - protein, 13 aa, Artificial Sequence
  Description of Artificial Sequence Synthetic peptide
LSSASRPHPVRST

SEQ ID NO: 6 - protein, 13 aa, Artificial Sequence
  Description of Artificial Sequence Synthetic peptide
VSPTYRHYPVTST

SEQ ID NO: 7 - protein, 29 aa, Artificial Sequence
  Description of Artificial Sequence Synthetic peptide
VSPTYRHYPVTSTCARRYFGVVADAFDIW

SEQ ID NO: 8 - protein, 29 aa, Artificial Sequence
  Description of Artificial Sequence Synthetic peptide
VSPTYRHYSVTSTCARRYFGVVADAFDIW

SEQ ID NO: 9 - protein, 4 aa, Artificial Sequence
  Description of Artificial Sequence Synthetic peptide
TCQQ

SEQ ID NO: 10 - protein, 13 aa, Artificial Sequence
  Description of Artificial Sequence Synthetic peptide
TCQQSYSTPPITF

SEQ ID NO: 11 - protein, 13 aa, Artificial Sequence
  Description of Artificial Sequence Synthetic peptide
ACQQSYSPPPITF

SEQ ID NO: 12 - protein, 17 aa, Artificial Sequence
  Description of Artificial Sequence Synthetic peptide
CALMGTYCSGDNCYSWF

SEQ ID NO: 13 - protein, 21 aa, Artificial Sequence
  Description of Artificial Sequence Synthetic peptide
SCALMGTYCSGDNCYSWFDPW

SEQ ID NO: 14 - protein, 21 aa, Artificial Sequence
  Description of Artificial Sequence Synthetic peptide
TCALMGTYCSGDNCYSWFDPW

SEQ ID NO: 15 - protein, 6 aa, Artificial Sequence
  Description of Artificial Sequence Synthetic peptide
VCQAWD

SEQ ID NO: 16 - protein, 12 aa, Artificial Sequence
  Description of Artificial Sequence Synthetic peptide
VCQAWDSSVVVF

SEQ ID NO: 17 - protein, 13 aa, Artificial Sequence
  Description of Artificial Sequence Synthetic peptide
KASNQGESSSSSV

SEQ ID NO: 18 - protein, 34 aa, Artificial Sequence
  Description of Artificial Sequence Synthetic polypeptide
KASNQGESSSSSVYCARDSWYSSGRNTPNWFDPW

SEQ ID NO: 19 - protein, 34 aa, Artificial Sequence
  Description of Artificial Sequence Synthetic polypeptide
TASNQGESSSSSVYCARDSWYSSGRNTPNWFDPW

SEQ ID NO: 20 - protein, 34 aa, Artificial Sequence
  Description of Artificial Sequence Synthetic polypeptide
KASNQGESSSSSVYCARDSWYTSGRNTPNWFDPW

SEQ ID NO: 21 - protein, 34 aa, Artificial Sequence
  Description of Artificial Sequence Synthetic polypeptide
KASNQDESSSSSVYCARDSWYSSGRNTPNWFDPW

SEQ ID NO: 22 - protein, 34 aa, Artificial Sequence
  Description of Artificial Sequence Synthetic polypeptide
KASNQGESSSSSLYCATDSWYSSGRNTPNWFDPW

SEQ ID NO: 23 - protein, 34 aa, Artificial Sequence
  Description of Artificial Sequence Synthetic polypeptide
KASDQGESSSSSLYCATDSWYSSGRNTPNWFDPW

SEQ ID NO: 24 - protein, 34 aa, Artificial Sequence
  Description of Artificial Sequence Synthetic polypeptide
KGSNQGESSSSCVYCARDSWYTSGRNTPNWFDPW

SEQ ID NO: 25 - protein, 34 aa, Artificial Sequence
  Description of Artificial Sequence Synthetic polypeptide
KASNHDESSSSSVYCARDSWYSSGRNTPNWFDPW

SEQ ID NO: 26 - protein, 34 aa, Artificial Sequence
  Description of Artificial Sequence Synthetic polypeptide
KASNQGDSTSSSVYCARDSWYSSGRNTPNWFDPW

SEQ ID NO: 27 - protein, 10 aa, Artificial Sequence
  Description of Artificial Sequence Synthetic peptide
KVYCQVWDSS

SEQ ID NO: 28 - protein, 17 aa, Artificial Sequence
  Description of Artificial Sequence Synthetic peptide
KVYCQVWDSSSDHPYVF

SEQ ID NO: 29 - protein, 17 aa, Artificial Sequence
  Description of Artificial Sequence Synthetic peptide
KVYCQVWDVSSDHPYVF

SEQ ID NO: 30 - protein, 17 aa, Artificial Sequence
  Description of Artificial Sequence Synthetic peptide
KVYCQVWDNSSDHPYVF

SEQ ID NO: 31 - protein, 17 aa, Artificial Sequence
  Description of Artificial Sequence Synthetic peptide
KVFCQVWDSSSDHPYVF

SEQ ID NO: 32 - protein, 17 aa, Artificial Sequence
  Description of Artificial Sequence Synthetic peptide
KVYCQVWNSSSDHPYVF

SEQ ID NO: 33 - protein, 11 aa, Artificial Sequence
  Description of Artificial Sequence Synthetic peptide
AGSIQYCYSTD

SEQ ID NO: 34 - protein, 19 aa, Artificial Sequence
  Description of Artificial Sequence Synthetic peptide
AGSIQYCYSTDSSGNLVVF

SEQ ID NO: 35 - protein, 19 aa, Artificial Sequence
  Description of Artificial Sequence Synthetic peptide
AGSIQYCYSADSTGNLVVF

SEQ ID NO: 36 - protein, 19 aa, Artificial Sequence
  Description of Artificial Sequence Synthetic peptide
AGRVQYCYSTDSSGNLVVF

SEQ ID NO: 37 - protein, 19 aa, Artificial Sequence
  Description of Artificial Sequence Synthetic peptide
TGSIQYCYSTDSSGNLVVF

SEQ ID NO: 38 - protein, 19 aa, Artificial Sequence
  Description of Artificial Sequence Synthetic peptide
TGSIQYCYSIDSSGNLVVF

SEQ ID NO: 39 - protein, 19 aa, Artificial Sequence
  Description of Artificial Sequence Synthetic peptide
AGSIRYCYSTDSSGNLVVF

SEQ ID NO: 40 - protein, 4 aa, Artificial Sequence
  Description of Artificial Sequence Synthetic peptide
WGDR

SEQ ID NO: 41 - protein, 20 aa, Artificial Sequence
  Description of Artificial Sequence Synthetic peptide
FAHTCARPKSDYIIDAFDIW

SEQ ID NO: 42 - protein, 20 aa, Artificial Sequence
  Description of Artificial Sequence Synthetic peptide
WAHTCARPKSDYIIDAFDIW

SEQ ID NO: 43 - protein, 8 aa, Artificial Sequence
  Description of Artificial Sequence Synthetic peptide
SSNCAAWD

SEQ ID NO: 44 - protein, 14 aa, Artificial Sequence
  Description of Artificial Sequence Synthetic peptide
NRSCAAWDDSLWVF

(19) United States
(12) Patent Application Publication
(10) Pub. No.: US 2021/0327544 A1
(43) Pub. Date: Oct. 21, 2021

(54) SYSTEMS AND METHODS FOR VISUALIZING ADAPTIVE IMMUNE CELL CLONOTYPING DATA

(71) Applicant: 10x Genomics, Inc., Pleasanton, CA (US)

(72) Inventors: David Jaffe, Pleasanton, CA (US); Sreenath Krishnan, San Jose, CA (US); Wyatt McDonnell, Pleasanton, CA (US); Michael Stubbington, Cambridge (GB)

(21) Appl. No.: 17/233,029

(22) Filed: Apr. 16, 2021

Related U.S. Application Data
(60) Provisional application No. 63/011,779, filed on Apr. 17, 2020.

(51) Int. Cl. G16B 45/00; G16B 25/10; G16B 30/00

(52) U.S. Cl. CPC G16B 45/00; G16B 30/00; G16B 25/10

(57) ABSTRACT

An interactive visualization system is disclosed herein. The system includes a data source, user input device, processor, and display. The data source obtains a B cell receptor and/or T cell receptor data source. The user input device receives a user selected parameter under which to analyze the data set. The processor identifies a clonotype group in the data set using the parameter, identifies subclonotypes within the clonotype group (wherein each identified subclonotype comprises cells having identical V(D)J transcripts), and processes the data to define a visualization model that can display a compressed view of the identified clonotype group. The display renders a visualization of said data set according to said visualization model. The visualization displays the clonotype group by identified subclonotype.

### CROSS-REFERENCE

The present application claims priority to U.S. Provisional Application No. 63/011,779, entitled “SYSTEMS AND METHODS FOR VISUALIZING ADAPTIVE IMMUNE CELL CLONOTYPING DATA,” filed on Apr. 17, 2020, which application is entirely incorporated herein by reference for all purposes.

### FIELD

This description is generally directed towards systems and methods for analyzing immune cell clonotype data generated using single- and multi-modal single cell genomic sequencing technologies. More specifically, there is a need for systems and methods to visualize and present immune cell clonotype data so that it is readily analyzed and interpreted by a user. Systems and methods to visualize and present these data for analysis and interpretation are useful and readily applied to data generated using non-droplet and droplet-based microfluidic single cell genomic sequencing technologies, array-based microwell- and nanowell-based single cell genomic sequencing technologies, in situ sequencing technologies, and spatially indexed single cell technologies.

### BACKGROUND

The immune system recognizes and eliminates non-self threats through a complex and layered network of both innate and adaptive immune cells. Robust characterization of this response and discovery of novel cell types and antigen-specific populations has proven challenging to perform in a high-throughput fashion due to the limited number of analytes that can be measured simultaneously using flow cytometry, CyTOF, and similar assays. One approach to addressing these limitations is to utilize multi-modal single cell technologies, such as microfluidic droplet-based single cell techniques. Applications of these technologies include the analysis of pre- and post-vaccination T cells, B cells, and peripheral blood mononuclear cells from influenza vaccines or other vaccines (or of samples collected from individuals affected by diseases such as systemic lupus erythematosus and other autoimmune disorders, chronic viral infection, and acute/non-chronic viral infection), or T cells/B cells/PBMCs from individuals treated with a drug or biological molecule such as a checkpoint inhibitor, anti-cancer drug, monoclonal antibody, or antibody-drug conjugate. Importantly, these single cell assays allow users to learn the full and paired sequences of heterodimeric and extremely polymorphic immune cell receptors of adaptive lymphocytes, e.g., T cells and B cells, and to identify from which single cell (and its corresponding phenotype, genotype, and antigen specificity) a given immune receptor had originated. This relationship is masked or not directly observable using bulk DNA and RNA-based sequencing assays and is not captured in a cost-effective or high-throughput fashion in plate-based assays.

Using this framework, vaccine-specific T cell and B cell responses can be identified and used to implement an immune cell (B cells/T cells/PBMCs) clonotyping algorithm that resolves post-vaccination, post-disease or post-treatment activated immune cell antibody lineages at scale by combining untargeted and targeted gene expression, full-length immune cell receptor sequencing, surface protein expression and/or antigen capture, in addition to tag-based and genetic demultiplexing.

As such, there is a need for systems and methods that can aid in the visualization, and presentation of immune cell clonotype data generated using single- and multi-modal single cell genomic sequencing technologies for analysis and interpretation.

### SUMMARY

In accordance with various embodiments, an interactive visualization system, is disclosed. The system includes a data source, user input device, processor, and display. The data source obtains a B cell receptor and/or T cell receptor data source. The user input device receives a user selected parameter under which to analyze the data set. The processor identifies a clonotype group in the data set using the parameter, identifies subclonotypes within the clonotype group (wherein each identified subclonotype comprises cells having identical V(D)J transcripts), and processes the data to define a visualization model that can display a compressed view of the identified clonotype group. The display renders a visualization of said data set according to said visualization model. The visualization displays the clonotype group by identified subclonotype.

In accordance with various embodiments, a method for interactively visualizing and examining clonotypes within single cell datasets, is disclosed. A B cell receptor and/or T cell receptor data set is obtained. A parameter under which to analyze the data set is received. A clonotype group in the data set is identified using the parameter. Subclonotypes within the clonotype group are identified. Each identified subclonotype comprises cells having identical V(D)J transcripts. The data is processed to define a visualization model that can display a compressed view of the identified clonotype group. A visualization of said data set according to said visualization model is rendered. The visualization displays the clonotype group by identified subclonotype.

In accordance with various embodiments, a graphical user interface (GUI) for displaying immune cell clonotyping information, is disclosed. The GUI includes a listing of subclonotypes of a immune cell clonotype. The subclonotypes share identical V(D)J transcripts, wherein the listing of subclonotypes includes a number of cells associated with each subclonotype. The GUI further includes a listing of one or more textual frames with information about chains common to each member of the immune cell clonotype. The textual frame contains an amino acid sequence for the variable and constant regions of each subclonotype. The GUI also includes a positional information for each member of the amino acid sequence.

These and other aspects and implementations are discussed in detail herein. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF FIGURES

The accompanying drawings are not intended to be drawn to scale. Like reference numbers and designations in the various drawings indicate like elements. For purposes of clarity, not every component may be labeled in every drawing. In the drawings:

FIG. 1 is an example visualization displaying immune cell clonotyping information, in accordance with various embodiments.

FIG. 2 is an example visualization displaying immune cell clonotyping information, in accordance with various embodiments.

FIG. 3 is an example visualization displaying immune cell clonotyping information, in accordance with various embodiments.

FIG. 4 illustrates is a block diagram of a computer system, in accordance with various embodiments.

FIG. 5 is an example visualization displaying immune cell clonotyping information, in accordance with various embodiments.

FIG. 6 illustrates an interactive visualization system, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

Unless defined otherwise, all technical and scientific terms used herein have the same meaning as commonly understood by one of ordinary skill in the art to which their various embodiments belong.

All publications mentioned herein are incorporated herein by reference for the purpose of describing and disclosing devices, compositions, formulations and methodologies which are described in the publication and which might be used in connection with the present disclosure.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronical-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ, NEXTSEQ, and NOVASEQ Systems of Illumina, the DNBSEQ and BGISEQ platforms of Beijing Genomics Institute (BGI), the GRIDION and PROMETHION Systems of Oxford Nanopore Technologies, PACBIO SEQUEL Systems of Pacific Biosciences, and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.), which denotes a single or a grouping of genes (in DNA or RNA) that have undergone changes as referenced against a particular species or sub-populations within a particular species due to mutations, recombination/crossover or genetic drift.

In general, the methods and systems described herein accomplish sequencing of nucleic acid molecules including, but not limited to, DNA (e.g., genomic DNA), RNA (e.g., mRNA, including full-length mRNA transcripts, and small RNAs, such as miRNA, tRNA, and rRNA), and cDNA. In various embodiments, the methods and systems described herein accomplish genomic sequencing of nucleic acid molecules (e.g., DNA, RNA, and mRNA). In various embodiments, the methods and systems described herein accomplish genomic sequencing of immune cell receptor sequences (e.g., DNA, RNA, and mRNA). In various embodiments, the methods and systems described herein can accomplish transcriptome sequencing, e.g., whole transcriptome sequencing of mRNA encoding immune cell receptors. In some embodiments, the methods and systems described herein can also accomplish targeted genomic sequencing of nucleic acid molecules (e.g., DNA, RNA, and mRNA). In various embodiments, the methods and systems described herein accomplish single cell genomic sequencing, for example, single cell genomic sequencing of nucleic acid molecules (e.g., RNA and mRNA) encoding immune cell receptors of single cells, such as B cell receptors (BCRs) and T cell receptors (TCRs).

In various embodiments, the methods and systems described herein can include high-throughput sequencing technologies, e.g., high-throughput DNA and RNA sequencing technologies. In various embodiments, the methods and systems described herein can include high-throughput, higher accuracy short-read DNA and RNA sequencing technologies. In various embodiments, the methods and systems described herein can include long-read RNA sequencing, e.g., by sequencing cDNA transcripts in their entirety without assembly. In various embodiments, the methods and systems described herein can also, for example, segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long-range molecular context is not only preserved through a sequencing process, but is also preserved through the targeted enrichment process used in targeted sequencing approaches.

In general, the methods and systems described herein are directed to single cell analysis (including single- and multi-modal analyses) of genomic sequencing of nucleic acids (e.g., RNA and mRNA) encoding immune cell receptors of single cells, such as B cell receptors (BCRs) and T cell receptors (TCRs). Single cell analysis, including single cell multi-modal analyses (e.g., single cell immune cell receptor sequencing combined with, for example, gene expression, protein expression, and/or antigen capture technologies), as well as processing and sequencing of nucleic acids, in accordance with the methods and systems described in the present application are described in further detail, for example, in U.S. Pat. Nos. 9,689,024; 9,701,998; 10,011,872; 10,221,442; 10,337,061; 10,550,429; 10,273,541; and U.S. Pat. Pub. 20180105808, which are all herein incorporated by reference in their entirety for all purposes and in particular for all written description, figures and working examples directed to processing nucleic acids and sequencing and other characterizations of genomic material.

The term “B cells”, also known as B lymphocytes, refer to a type of white blood cell of the small lymphocyte subtype. They function in the humoral immunity component of the adaptive immune system by expressing and/or secreting antibodies. Additionally, B cells present antigens (they are also classified as professional antigen-presenting cells (APCs)) and secrete cytokines. In mammals, B cells mature in the bone marrow, which is at the core of most bones. In birds, B cells mature in the bursa of Fabricius, an immune organ where they were first discovered by Chang and Glick, (B for bursa) and not from bone marrow as commonly believed. B cells, unlike the other two classes of lymphocytes, T cells and natural killer cells, express B cell receptors (BCRs) on their cell membrane or secrete their BCRs if they have differentiated into long-lived plasma cells. BCRs allow a B cell to bind to specific antigens, against which it will initiate an antibody response.

The term “T cell”, also known as T lymphocytes, refer to a type of an adaptive immune cell. T cells develops in the thymus gland, hence the name T cell, and play a central role in the immune response of the body. T cells can be distinguished from other lymphocytes by the presence of a T cell receptor (TCR) on the cell surface. These immune cells originate as precursor cells, derived from bone marrow, and then develop into several distinct types of T cells once they have migrated to the thymus gland. T cell differentiation continues even after they have left the thymus. T cells include, but are not limited to, helper T cells, cytotoxic T cells, memory T cells, regulatory T cells, and killer T cells. Helper T cells stimulate B cells to make antibodies and help killer cells develop. Based on the T cell receptor chain, T cells can also include T cells that express αβ TCR chains, T cells that express γδ TCR chains, as well as unique TCR co-expressors (i.e., hybrid αβ-γδ T cells) that co-express the αβ and γδ TCR chains.

T cells can also include engineered T cells that can attack specific cancer cells. A patient's T cells can be collected and genetically engineered to produce chimeric antigen receptors (CAR). These engineered T cells are called CAR T cells, which forms the basis of the developing technology called CAR-T therapy. These engineered CAR T cells are grown by the billions in the laboratory and then infused into a patient's body, where the cells are designed to multiply and recognize the cancer cells that express the specific protein. This technology, also called adoptive cell transfer is emerging as a potential next-generation immunotherapy treatment.

T cells, such as the killer T cells can directly kill cells that have already been infected by a foreign invader. T cells can also use cytokines as messenger molecules to send chemical instructions to the rest of the immune system to ramp up its response. Activating T cells against cancer cells is the basis behind checkpoint inhibitors, a relatively new class of immunotherapy drugs that have recently been approved to treat lung cancer, melanoma, and other difficult cancers. Cancer cells often evade patrolling T cells by sending signals that make them seem harmless. Checkpoint inhibitors disrupt those signals and prompt the T cells to attack the cancer cells.

The term “naïve”, as used herein, can refer to B-lymphocytes or T-lymphocytes that have not yet reacted with an epitope of an antigen or that have a cellular phenotype consistent with that of a lymphocyte that has not yet responded to antigen-specific activation after clonal licensing.

The term “Fab”, also referred to as an antigen-binding fragment, refers to the variable portions of an antibody molecule with a paratope that enables the binding of a given epitope of a cognate antigen. The amino acid and nucleotide sequences of the Fab portion of antibody molecules are hypervariable. This is in contrast to the “Fc” or crystallizable fragment, which is relatively constant and encodes the isotype for a given antibody; this region can also confer additional functional capacity through processes such as antibody-dependent complement deposition, cellular cytotoxicity, cellular trogocytosis, and cellular phagocytosis.

The phrase “clonal selection” refers to the selection and activation of specific B lymphocytes and T lymphocytes by the binding of epitopes to B cell receptors or T cell receptors with a corresponding fit and the subsequent elimination (negative selection) or licensing for clonal expansion (positive selection) of a B or T lymphocyte after binding of an antigenic determinant.

The phrase “clonal expansion” refers to the proliferation of B lymphocytes and T lymphocytes activated by clonal selection in order to produce a clonal population of daughter cells with the same antigen specificity and functional capacity. In the case of T lymphocytes this antigen specificity is exact at the nucleotide and protein level and in the case of B lymphocytes this antigen specificity can be exact at the nucleotide and protein level or mutated relative to the parent population by mutations at the nucleotide level (and by extension the protein level). This enables the body to have sufficient numbers of antigen-specific lymphocytes to mount an effective immune response.

The term “cytokines” refers to a wide variety of intercellular regulatory proteins produced by many different cells in the body, which ultimately control every aspect of body defense. Cytokines activate and deactivate phagocytes and immune defense cells, enhance or inhibit the functions of the different immune defense cells, and promote or inhibit a variety of nonspecific body defenses.

The phrase “T helper lymphocytes”, also referred to as helper cells, refer to a type of white blood cell that orchestrate the immune response and enhance the activities of the killer T-cells (those that destroy pathogens) and B cells (antibody and immunoglobulin producers).

The phrase “affinity maturation” refers to the gradual modification of the paratope and entire B cell receptor as a result of somatic hypermutation. B lymphocytes with higher affinity B cell receptors that can 1) bind the epitope more tightly and 2) therefore bind the epitope for a longer period of time are able to proliferate more and survive longer. These B cells can eventually differentiate into plasma cells, which secrete their antibodies and form the basis of serum-mediated immunity.

The phrase “somatic hypermutation” (SHM) refers to a cellular mechanism by which the adaptive immune system adapts to foreign elements confronting it (e.g. viruses, bacteria, biomolecules). A major component of the process of affinity maturation, SHM diversifies B cell receptors used to recognize foreign elements (antigens) and allows the immune system to adapt its response to new threats during the lifetime of an organism. Somatic hypermutation involves a programmed process of mutation predominantly affecting select framework and complementarity-determining regions of immunoglobulin genes. Unlike germline mutation, SHM operates at the level of an organism's individual immune cells. These mutations are not transmitted to the organism's offspring, but are transmitted to daughter cells of individual B cell clones. Mistargeted somatic hypermutation is a likely mechanism in the development of B cell lymphomas and many other cancers. Somatic hypermutation can also lead to the acquisition of non-VDJ template DNA within B cell receptor sequences, such as LAIR1 insertions in malaria-specific neutralizing antibodies.

Somatic hypermutation is a distinct diversification mechanism from isotype switching (also called class switching). Mutations acquired during somatic hypermutation eventually lead to isotype switching, in which a B cell's antibody can be coupled to different functions by switching to a different Fc/constant region sequence. Isotype switching is an irreversible process, in that once a B cell has switched from a given constant region (e.g. IGHM) to a new constant region (e.g. IGHA1) it can no longer use the IgM constant region as the DNA encoding the IgM Fc is excised and removed during isotype switching.

The term “contig”, originating from the term “contiguous”, refers to a set of overlapping DNA segments that together represent a consensus region of DNA. In bottom-up sequencing projects, a contig refers to overlapping sequence data (reads); in top-down sequencing projects, contig refers to the overlapping clones that form a physical map of the genome that is used to guide sequencing and assembly. Contigs can thus refer both to overlapping DNA sequences and to overlapping physical segments (fragments) contained in clones depending on the context. Note that clone, in reference to overlapping clones, refers to individual bacteria or constructs (e.g. phagemids, cosmids, etc.) containing distinct insertions of genomes that were utilized in early efforts to map genomes

The phrase “heavy chain” refers to the large polypeptide subunit of an antibody (immunoglobulin). The first recombination event to occur is between one D and one J gene segment of the heavy chain locus. Any DNA between these two gene segments is deleted. This D-J recombination is followed by the joining of one V gene segment, from a region upstream of the newly formed DJ complex, forming a rearranged VDJ gene segment. All other gene segments between V and D segments are now deleted from the cell's genome. Primary transcript (unspliced RNA) is generated containing the VDJ region of the heavy chain and both the constant mu and delta chains (Cμ and Cδ) (i.e., the primary transcript contains the segments: V-D-J-Cμ-Cδ). The primary RNA is processed to add a polyadenylated (poly-A) tail after the Cμ chain and to remove sequence between the VDJ segment and this constant gene segment. Translation of this mRNA leads to the production of the IgM heavy chain protein and the IgD heavy chain protein (its splice variant). Expression of the immunoglobulin heavy chain with one or more surrogate light chains constitutes the pre-B cell receptor that allows a B cell to undergo selection and maturation.

The phrase “light chain” refers to the small polypeptide subunit of an antibody (immunoglobulin). The kappa (κ) and lambda (λ) chains of the immunoglobulin light chain loci rearrange in a very similar way, except that the light chains lack a D segment. In other words, the first step of recombination for the light chains involves the joining of the V and J chains to give a VJ complex before the addition of the constant chain gene during primary transcription. Translation of the spliced mRNA for either the kappa or lambda chains results in formation of the Igκ or Igλ light chain protein. Assembly of the Igμ heavy chain and one of the light chains results in the formation of membrane bound form of the immunoglobulin IgM that is expressed on the surface of the immature B cell. B cells may express up to two heavy chains and/or two light chains in respectively rare and uncommon instances through a phenomenon known as allelic inclusion. This phenomenon can only be directly observed using single-cell technologies, though it can be inferred with a degree of uncertainty using a combination of bulk sequencing technologies and probabilistic inference via an extension of the birthday paradox.

The phrase “complementarity-determining regions” (CDRs) refers to part of the variable chains in immunoglobulins (antibodies) and T cell receptors, generated by B cells and T cells respectively, where these molecules are particularly hypervariable. The antigen-binding site of most antibodies and T cell receptors is typically distributed across these CDRs, collectively forming a paratope. However, there are many documented examples of paratopes that enable antigen recognition that fall outside of the CDRs. As the most variable parts of the molecules, CDRs are crucial to the diversity of antigen specificities and immune cell receptor sequences generated by lymphocytes.

V(D)J recombination is a genetic recombination mechanism that occurs in developing lymphocytes during the early stages of T and B cell maturation. Through somatic recombination, this mechanism produces a highly diverse repertoire of antibodies/immunoglobulins and T cell receptors (TCRs) found in B cells and T cells, respectively. This process is a defining feature of the adaptive immune system and these receptors are defining features of adaptive immune cells.

V(D)J recombination occurs in the primary immune organs (bone marrow for B cells and thymus for T cells) and in a generally random fashion. The process leads to the rearranging of variable (V), joining (J), and in some cases, diversity (D) gene segments. As discussed above, the heavy chain possesses numerous V, D, and J gene segments, while the light chain possesses only V and J gene segments. The process ultimately results in novel amino acid sequences in the antigen-binding regions of immunoglobulins and TCRs that allow for the recognition of antigens from nearly all pathogens including, for example, bacteria, viruses, and parasites. Furthermore, the recognition can also be allergic in nature or may recognize host tissues and lead to autoimmunity.

Human antibody molecules, including B cell receptors (BCRs), include both heavy and light chains, each of which contains both constant (C) and variable (V) regions, and are genetically encoded on three loci. The first is the immunoglobulin heavy locus on chromosome 14, containing the gene segments for the immunoglobulin heavy chain. The second is the immunoglobulin kappa (κ) locus on chromosome 2, containing the gene segments for part of the immunoglobulin light chain. The third is the immunoglobulin lambda (λ) locus on chromosome 22, containing the gene segments for the remainder of the immunoglobulin light chain.

Each heavy or light chain contains multiple copies of different types of gene segments for the variable regions of the antibody proteins. For example, the human immunoglobulin heavy chain region contains two C gene segments (Cμ and Cδ), 44 V gene segments, 27 D gene segments and 6 J gene segments. The number of given segments present in any individual can vary, as these gene segments are carried in haplotypes; for this reason, inference of both the alleles present within any individuals and the germline sequence of those alleles is an important step in correctly identifying B cell clonotypes. The light chains possess two C gene segments (Cλ and Cκ) and numerous V and J gene segments, but do not have D gene segments. DNA rearrangement causes one copy of each type of gene segment to mate with any given lymphocyte, generating a substantial antibody repertoire. Approximately 10¹⁴ combinations are possible, with 1.5×10² to 3×10³ potentially removed via self-reactivity.

Accordingly, each naïve B cell makes an antibody with a unique Fab site through a series of gene recombinations, and later mutations, with the specific molecules of the given antibody attaching to the B cell's surface as a B cell receptor (BCR). These BCRs are then available to react with epitopes of an antigen.

When the immune system encounters an antigen, epitopes of that antigen will be presented to many B lymphocytes. B lymphocytes must first rearrange a heavy chain that enables pre-B cell receptor ligand binding. B lymphocytes that bind multivalent self-targets after rearrangement of the light chain too strongly are eliminated and die or undergo a secondary recombination event, while B cells that do not bind self-targets too strongly are licensed to exit the bone marrow. The latter becomes available to respond to non-self antigens and to undergo clonal expansion. This process is known as clonal selection.

Cytokines produced by activated CD4 T helper lymphocytes enable those activated B lymphocytes (B cells) to rapidly proliferate to produce large clones of thousands of identical B cells. More specifically, when under threat (i.e., via bacteria, virus, etc.), the body releases white blood cells by the immune system. CD4 T lymphocytes help the response to a threat by triggering the maturation of other types of white blood cell. They produce special proteins, called cytokines, have plural functions, including the ability to summon all of the other immune cells to the area, and also the ability to cause nearby cells to differentiate (become specialized) into mature B cells and T cells.

Accordingly, while only a few B cells in the body may have an antibody molecule that can bind a particular epitope, eventually many thousands of cells are produced with the right specificity, allowing the body's immune system to act en masse. This is referred to as clonal expansion. Natural phenomena such as IgA deficiency and murine transgenic models have shown that there are multiple paths by which a B cell receptor can acquire novel antigen specificity even from a very limited repertoire through the processes of somatic hypermutation and affinity maturation.

As the B cells proliferate, they undergo affinity maturation as a result of somatic hypermutation. This allows the B cells to “fine-tune” the paratopes of the antibody to more effectively fit with the recognized epitopes. B cells with high affinity B cell receptors on their surface bind epitopes more tightly and for a longer period of time, which enables these cells to selectively proliferate. Over the course of this proliferation and expansion, these variant B cells differentiate into plasma cells that synthesize and secrete vast quantities of antibodies with Fab sites that fit the target epitopes very precisely.

The phrase “immune cell” refers to a cell that is part of the immune system and that helps the body fight infections and other diseases. Immune cells include innate immune cells (such as basophils, dendritic cells, neutrophils, etc.) that are the first line of body's defense and are deployed to help attack the invading foreign cells (e.g., cancer cells) and pathogens. The innate immune cells can quickly respond to foreign cells and pathogens to fight infection, battle a virus, or defend the body against bacteria. Immune cells can also include adaptive immune cells (such as lymphocytes including B cells and T cells). The adaptive immune cells can come into action when an invading foreign cells or pathogens slip through the first line of body's defense mechanism. The adaptive immune cells can take longer to develop, because their behaviors evolve from learned experiences, but they can tend to live longer than innate immune cells. Adaptive immune cells remember foreign invaders after their first encounter and fight them off the next time they enter the body. Both types of immune cells employ important natural defenses in helping the body fight foreign cells and pathogens for fighting infections and other diseases.

Accordingly, the immune cells of the disclosure can include, but are not limited to, neutrophils, eosinophils, basophils, mast cells, monocytes, macrophages, dendritic cells, natural killer cells, and lymphocytes (such as B cells and T cells). The immune cells of the disclosure can further include dual expresser cells or DE (such as unique dual-receptor-expressing lymphocytes that co-express functional B cell receptor (BCR) and T cell receptor (TCR)), cells with adaptive immune receptors that may diversify or may not diversify (including immune cells expressing a chimeric antigen receptor with a fixed nucleotide sequence or with the capacity to mutate), and TCR co-expressors (i.e., hybrid αβ-γδ T cells) that co-express both αβ and γδ TCR chains.

The phrase “immune cell receptor”, “immune receptor”, or “immunologic receptor” refers to a receptor or immune cell receptor sequence, usually on a cell membrane, which can recognize components of pathogenic microorganisms (e.g., components of bacterial cell wall, bacterial flagella or viral nucleic acids) and foreign cells (e.g., cancer cells), which are foreign and not found naturally on the host cells, or binds to a target molecule (for example, a cytokine), and causes a response in the immune system. The immune cell receptors of the immune system can include, but are not limited to, pattern recognition receptors (PRRs), Toll-like receptors (TLRs), killer activated and killer inhibitor receptors (KARs and KIRs), complement receptors, Fc receptors, B cell receptors, and T cell receptors.

The phrase “immune cell receptor sequences” of an immune cell receptor include both heavy and light chains, each of which contains both constant (C) and variable (V) regions. For example, B cell receptors (BCRs) or B cell receptor sequences (including human antibody molecules) comprise of immunoglobulin heavy and light chains, each of which contains both constant (C) and variable (V) regions. Each heavy or light chain not only contains multiple copies of different types of gene segments for the variable regions of the antibody proteins, but also contains constant regions. For example, the BCR or human immunoglobulin heavy chain contains two (2) constant (Constant mu (Cμ) and delta (Cδ)) gene segments and forty-four (44) Variable (V) gene segments, plus twenty seven (27) Diversity (D) gene segments, and six (6) Joining (J) gene segments. The BCR light chains also possess two (2) constant gene segments ((Constant lambda (Cλ) and kappa (Cκ) and numerous V and J gene segments, but do not have any D gene segments. DNA rearrangement (i.e., recombination events) in developing B cells can cause one copy of each type of gene segment to go in any given lymphocyte, generating an enormous antibody repertoire. Accordingly, the primary transcript (unspliced RNA) of a BCR heavy chain can be generated containing the VDJ region of the heavy chain and both the constant mu and delta chains (Cμ and Cδ), i.e., the heavy chain primary transcript can contain the segments: V-D-J-Cμ-Cδ). In case of the B cell receptor and human immunoglobulin light chain, the first step of recombination for the light chains involves the joining of the V and J chains to give a VJ complex before the addition of the constant chain gene during primary transcription. Translation of the spliced mRNA for either the constant κ (Cκ) or λ (Cλ) chains results in formation of the Ig κ or Igλ light chain protein.

In general, most T cell receptors (TCR) are composed of an alpha (α) chain and a beta (β) chain, each of which contains both constant (C) and variable (V) regions. Thus, the most common type of a T cell receptor is called an alpha-beta TCR because it is composed of two different chains, one α-chain and one beta β-chain. A less common type of TCR is the gamma-delta TCR, which contains a different set of chains, one gamma (γ) chain and one delta (δ) chain. The T cell receptor genes are similar to immunoglobulin genes for the BCR and undergo similar DNA rearrangement (i.e., recombination events) in developing T cells as for the B cells. For example, the alpha-beta TCR genes also contain multiple V, D, and J gene segments in their beta chains and V and J gene segments in their alpha chains, which are re-arranged during the development of the T cells to provide a cell with a unique T cell antigen receptor. Thus, the β-chain of the TCR can contain Vβ-Dβ-Jβ gene segments and constant domain (Cβ) genes resulting in a Vβ-Dβ-Jβ-Cβ sequence of the TCR β-chain. The re-arrangement of the alpha (α) chain of the TCR follows β chain rearrangement, and can include Vα-Jα gene segments and constant domain (Cα) genes resulting in a Vα-Jα-Cα sequence of the TCR α-chain. Similar to the alpha-beta TCRs, the TCR-γ chain is produced by V-J recombinations and can contain Vγ-Jγ gene segments and constant domain (Cγ) genes resulting in a Vγ-Jγ-Cγ sequence of the TCR γ-chain, while the TCR-δ chain is produced using V-D-J recombinations, and can contain Vδ-Dδ-Jδ gene segments and constant domain (Cδ) genes resulting in a Vδ-Dδ-Jδ-Cδ sequence of the TCR δ-chain.

The phrase “immune cell receptor constant region sequence” or “immune receptor constant region sequence” refers to the constant region or constant region sequence of an immune cell receptor. For example, the immune cell receptor constant region sequence or immune receptor constant region sequence can include, but is not limited to, the constant mu (Cμ) and delta (Cδ) region genes and sequences of a BCR and immunoglobulin heavy chain, the constant lambda (Cλ) and kappa (Cκ) region genes and sequences of a BCR and immunoglobulin light chain, the alpha constant (Cα) region genes and sequences of a TCR α-chain sequence, the beta constant (Cβ) region genes and sequences of a TCR β-chain sequence, the gamma constant (Cγ) region genes and sequences of a TCR γ-chain sequence, and the delta constant (Cδ) region genes and sequences of a TCR δ-chain sequence.

With this understanding of the immune cell's purpose in fighting off attacking foreign antigens, the pharmaceutical industry has strongly focused on designing vaccines with the ability to expand antibody lineages directed towards specific B cells with shared antigen specificity. To most effectively determine the efficacy of a vaccine or antitumor antibody therapy, it is essential to be able to accurately identify cell members of a clonotype, which potentially share common or similar BCRs or antigen specificity. The pharmaceutical industry has also directed its efforts to isolate antibodies and antibody lineages against non-foreign targets for the purpose of developing antibody-based therapeutics for a broad array of disease states including autoimmune disease (anti-inflammatory targets), cancer (checkpoint inhibitors and other targets), and other conditions such as osteoporosis. Similarly, knowing the fine specificities of different antibody lineages elicited by a vaccine is essential to understanding serum neutralization profiles and global epitope maps of an entire virus. This same concept applies to understanding how a patient's adaptive immune system can render drugs such as adalimumab ineffective through the emergence of anti-drug antibodies and distinct anti-drug antibody lineage.

To understand what constitutes members of a clonotype, one can start with the original progenitor cell for a given lineage of B cells, this progenitor cell commonly referred to as the parent clone, which is a single cell to which all daughter cells will be genetically related, though their B cell receptors and exact antigen specificity may differ and diverge over time. Collectively, this parent clone and all its daughter cells constitute a clonotype. As stated above, accurate identification of the members of a clonotype is critical not just from a biological perspective, but also from the biomedical perspective, as correct identification of all of the members of a given clonotype can be useful in the design of vaccines (e.g., which antibody lineages can be expanded by a vaccine or are expanded successfully or unsuccessfully by a vaccine), in the monitoring of B cell-mediated immune disease (e.g., myasthenia gravis, lupus, B cell lymphoma), and in other settings (what antibodies are found in the tumor microenvironment or other immune niches during clinical disease). Known approaches that attempt to group immune cell receptor sequences into groups with shared antigen specificity or members of the same clonotype include, but are not limited to: immcantation, Clonify, GLIPH, TCRdist, VDJTools, MiXCR, AbSolve, and the algorithms described in PMID: 23536288, PMID: 23898164, PMID: 25345460, etc. While some of these algorithms can successfully identify groups of T cells with shared antigen specificity using single-cell data (TCRdist, GLIPH), and the other algorithms use solely bulk receptor sequencing data (i.e., without access to heavy and light chain sequences), none of these algorithms attempt to approximate the true clonotypes for B cells while also attempting to mitigate for sources of noise in the data nor while using the additional specificity found in the antibody light chain. Antibody discovery efforts have shown that false-positive antibody candidates are more frequently found in randomly paired antibody libraries than in natively paired antibody libraries, demonstrating the importance of correct clonotype identification from both biological and pharmaceutical perspectives. Further, none of these approaches provide easy visualization and data interaction routines to display a large amount of information about the single cells within a clonotype in a compact and readily interpretable display.

Therefore, in accordance with various embodiments, various systems and methods are provided that display large amounts of information related to true clonotype groupings for B cells in a dynamic, interactive and compact graphical user interface (GUI). In accordance with various embodiments, a method is provided for interactively visualizing and examining clonotypes within single cell datasets. The method can comprise obtaining an immune cell (e.g., B-cell receptor, etc.) dataset, receiving a set of parameters under which to analyze the dataset, and identifying one or more clonotype groups in the data set using the parameters. The method can further comprise identifying subclonotypes within the clonotype group, wherein each identified subclonotype comprises cells having identical V(D)J transcripts, processing the data to define a visualization model that can display a compressed view of the identified clonotype group, and rendering a visualization of said data set according to said visualization model, wherein the visualization displays the clonotype group by identified subclonotype.

As there are only so many letters (represented bases or amino acids) that can be view in a row before the GUI becomes visually overwhelming, the letters/positions that are variable are displayed within a clonotype, hence, horizontal compaction. Since each subclonotype is comprised of a set of one or more cells. Inclusion of additional data to display, such as gene expression, antigen capture, surface protein/antibody capture, etc. could be used to display this data for each cell rather than a single line with summary statistics for a subclonotype. We do the latter in order to promote vertical compaction.

In accordance with various embodiments, the parameter can be a first parameter, the visualization model is a first visualization model, and the visualization is a first visualization, the method further comprising receiving a second parameter under which to analyze the data set, re-identifying a clonotype in the data set using the second parameter, and re-identifying subclonotypes within the clonotype, wherein each identified subclonotype comprises cells having identical V(D)J transcripts. The method can further comprise re-processing the data to define a second visualization model that can display a modified compressed view of the identified clonotype, and re-rendering a second visualization of said data set according to said second visualization model, wherein the second visualization displays a modified version of the clonotype by identified subclonotype.

In accordance with various embodiments, the visualization can include a comparison of at least one reference sequence to a subclonotype. The at least one reference sequence can include a reference sequence listing selected from the group consisting of a universal reference sequence or a user-supplied reference sequence, a donor reference sequence, and combinations thereof.

In accordance with various embodiments, the visualization can include a listing of amino acid differences between each subclonotype within a clonotype. In accordance with various embodiments, the visualization can include a listing of nucleotide differences between each subclonotype within a clonotype. In accordance with various embodiments, the visualization can include subclonotype information selected from the group consisting of gene expression, Hamming distance, Levenshtein distance or similar edit distance, antibody counts, antigen counts, CRISPR guide or directly captured feature counts, and combinations thereof. The gene expression subclonotype information can be selected from the group consisting of median gene expression, maximum gene expression, mean gene expression, and combinations thereof. The gene expression subclonotype information is reported as a UMI count. Median, maximum, mean, and similar summary statistics thereof can also be used in accordance with various embodiments to visualize and report the aforementioned features in addition to gene expression. Those knowledgeable in the art recognize that there are many additional such features that could be reported such as percentage of a given set of features within a single cell and other user-provided annotations for a set of single cells such as manual annotation or description of information relevant to one or more subclonotypes, as specified in a variety of file formats.

In accordance with various embodiments, for each subclonotype, the visualization can include chain-specific subclonotype information selected from the group consisting of V(D)J UMI count, V(D)J read count, constant region name, complementarity-determining region (CDR) sequence for any of CDR1/CDR2/CDR3, constant sequence length, 5′UTR sequence length, differences from a universal reference constant region, differences from the 5′UTR sequence, base differences between subclonotypes, framework region amino acid and nucleotide sequences and lengths for any of FWR1/FWR2/FWR3/FWR4, and combinations thereof.

In accordance with various embodiments, the method can further include receiving a user input including information configured to customize the visualization with information relevant to one or more clonotypes, one or more subclonotypes, one or more barcodes, or combinations thereof.

In accordance with various embodiments, a GUI is provided for displaying immune cell clonotyping information. The GUI can include a listing of subclonotypes of an immune cell clonotype, wherein the subclonotypes share identical V(D)J transcripts, wherein the listing of subclonotypes includes a number of cells associated with each subclonotype. The GUI can further include a listing of one or more textual frames with information about chains common to each member of the immune cell clonotype, wherein the textual frame contains an amino acid sequence for the variable and constant regions of each subclonotype. The GUI can further include a positional information for each member of the amino acid sequence. In accordance with various embodiments the nucleotide sequences and accompanying positional information for the variable and constant regions of each subclonotype can be displayed in place of or in parallel to the amino acid sequences for these regions.

In accordance with various embodiments, the listing of one or more textual frames can comprise two or more textual frames. In accordance with various embodiments, the listing of one or more textual frames can comprise two textual frames. In accordance with various embodiments, the listing of one or more textual frames can comprise three textual frames. It should be understood, however, that the listing of textual frames can include any number of textual frames as long as it is renderable on a computer display in a manner that can be navigated by a user.

In accordance with various embodiments, the listing of one or more textual frames can include a comparison of at least one reference sequence to a subclonotype. The at least one reference sequence can include a reference sequence listing selected from the group consisting of a universal reference sequence or user-supplied reference, a donor reference sequence, and combinations thereof. In accordance with various embodiments, the listing of one or more textual frames includes a listing of amino acid differences between each subclonotype within a clonotype. In accordance with various embodiments, the listing of one or more textual frames includes a listing of nucleotide differences between each subclonotype within a clonotype.

In accordance with various embodiments, the listing of subclonotypes includes subclonotype information selected from the group consisting of gene expression, Hamming distance, Levenshtein distance or similar edit distance, antibody counts, antigen counts, CRISPR guide or directly captured feature counts, and combinations thereof. The gene expression subclonotype information can be selected from the group consisting of median gene expression, maximum gene expression, mean gene expression, and combinations thereof. The gene expression subclonotype information can be reported as a UMI count for each cell belonging to a given exact subclonotype; the features listed above can also be reported in this fashion. These features can also be reported as percentages of a library, as a score or percentile or normalized value calculated elsewhere, or as a value from a matrix or appropriately formatted dataset that provides this information for each cell or for each set of cells within a clonotype or exact subclonotype.

In accordance with various embodiments, for each subclonotype, the textual frame can provide chain-specific subclonotype information selected from the group consisting of V(D)J UMI count, V(D)J read count, constant region name, complementarity-determining region (CDR) sequences for any of the CDR1/CDR2/CDR3 regions, constant sequence length, 5′UTR sequence length, differences from a universal reference constant region, differences from the 5′ UTR sequence, base differences between subclonotypes, framework region amino acid and nucleotide sequences and lengths for any of FWR1/FWR2/FWR3/FWR4, and combinations thereof.

In accordance with various embodiments, the GUI can further include a user input to receive information configured to customize the display of immune cell clonotyping information relevant to one or more clonotypes, exact subclonotypes, or barcodes.

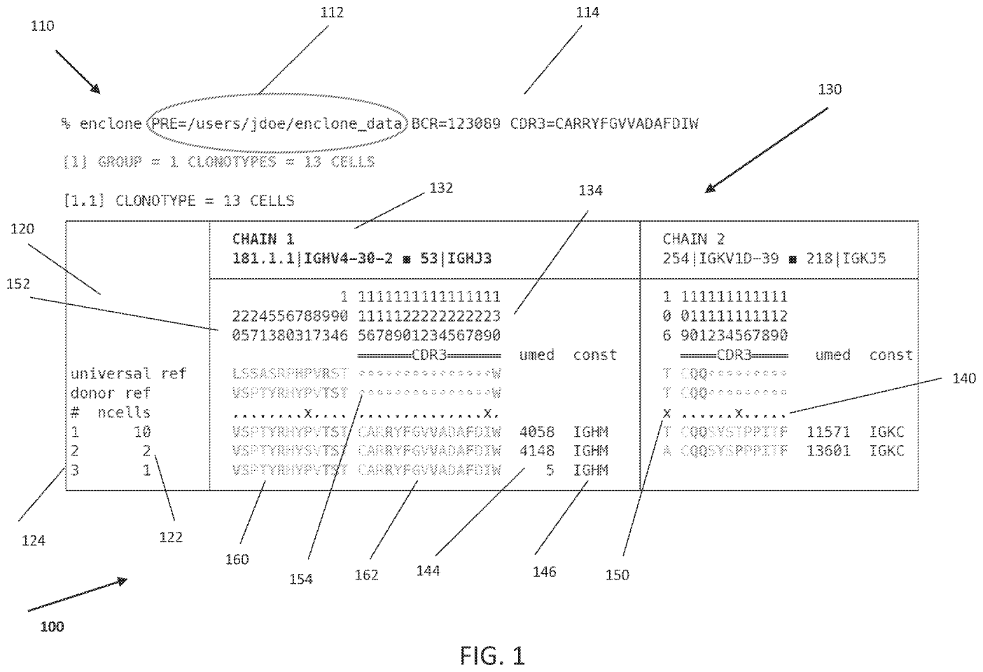

Referring to FIG. 1, an example visualization 100 of identified clonotypes is provided, in accordance with various embodiments. It should be noted that many details about the display features, fields, parameters, customizations, etc. are discussed below as opposed to this discussion of the visualizations of FIGS. 1-3 and 5. It should be understood, however, that while many of these details are discussed below rather than here, the display features, fields, parameters, customizations, etc., and the associated descriptions are relevant to all embodiments herein and can be implemented in any combination as per user need.

Returning to FIG. 1, visualization 100 can include a command line 110 that can be used for accepting a user input, in accordance with various embodiments. That user input can be, for example, a file path 112 to a dataset, and additional optional parameters 114 for customizing the output in visualization 100. As will be discussed below, specifying data sets can be done various ways including, for example, on the command line (as illustrated) via a supplementary metadata file. In the example visualization 100, the command line includes BCR and CDR3 parameters. Based on this example command line entry, the output visualization would exhibit all clonotypes in which at least one chain has the given CDR3 sequence. The output can be in a compressed view (e.g., streamlined visualization of query results to include essential information for specific analytical purposes).

Visualization 100 can include a grouping statement 114, which can include information such as, for example, the number of clonotype groups (one in FIG. 1), the number of clonotypes in the noted group (one in FIG. 1), and the number of cells in the noted clonotype (13 in FIG. 1). Clonotypes can be grouped into similar families having putatively similar function, with the grouping done automatically or via user-specified filters. These filters can include collapsing clonotypes based on V gene, similarity across the CDR3/junction sequence or the full-length heavy and/or light chains, reporting of singleton chains matching higher-frequency subclonotypes, detection and identification of indels within subclonotypes, and more. In accordance with various embodiments, the display can conceptually distinguish between clonotypes (e.g., as evolutionary families) and clonotype groups (e.g., as functional families).

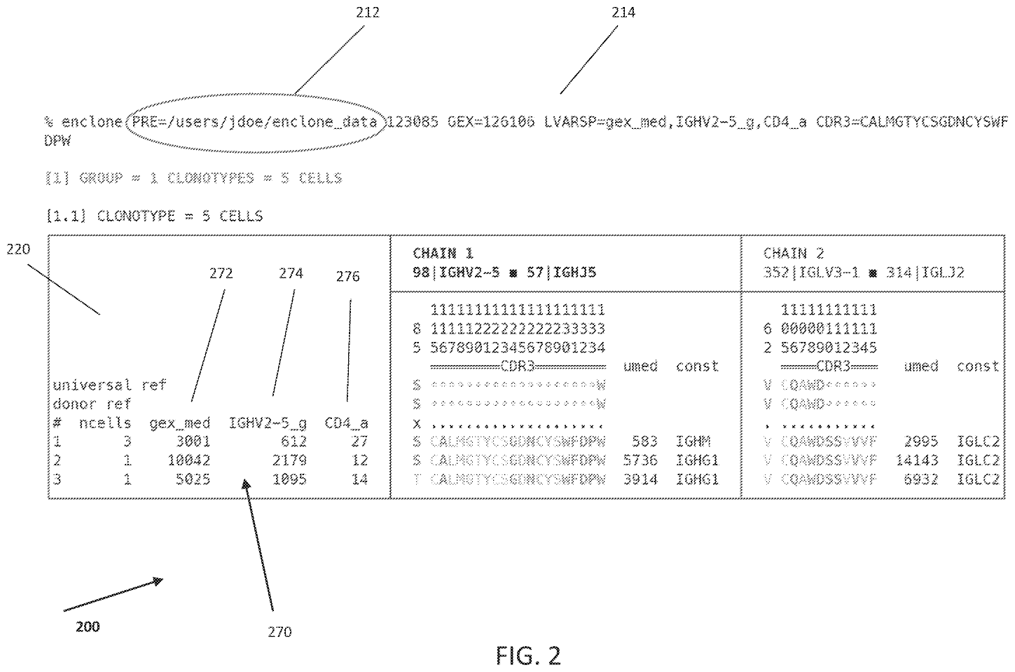

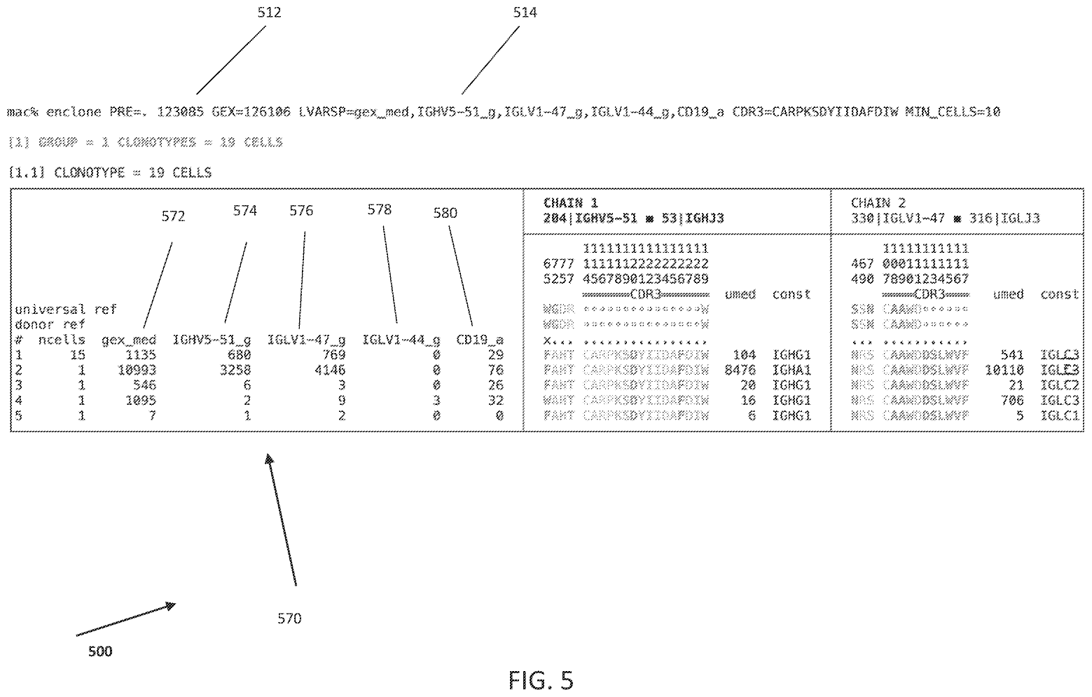

As discussed above, visualization 100 can also include a subclonotypes listing frame 120 for an immune cell clonotype, in accordance with various embodiments. The subclonotypes can share identical V(D)J transcripts. The listing of subclonotypes can include a number of cells 122 associated with each subclonotype (or exact subclonotype). Each line of frame 120 can be configured to represent an exact subclonotype 124, which is, as discussed in more detail herein, a set of cells having identical V(D)J transcripts. As discussed in detail herein, the columns in the subclonotypes listing frame 120 are configurable and can include many different types of information (discussed in detail below), some of which are illustrated in FIGS. 2 and 5, discussed below.

Further, the subclonotypes listing frame 120 can include subclonotype information selected from the group consisting of gene expression, Hamming distance, antibody, and combinations thereof. The gene expression subclonotype information can be selected from the group consisting of median gene expression, maximum gene expression, mean gene expression, and combinations thereof. The gene expression subclonotype information can be reported as a UMI count. These listing frame options are more evident in FIGS. 2 and 5. Median, maximum, mean, and similar summary statistics thereof can also be used in accordance with various embodiments to visualize and report the aforementioned features in addition to gene expression. Those knowledgeable in the art recognize that there are many additional such features that could be reported such as percentage of a given set of features within a single cell and other user-provided annotations for a set of single cells such as manual annotation or description of information relevant to one or more subclonotypes, as specified in a variety of file formats

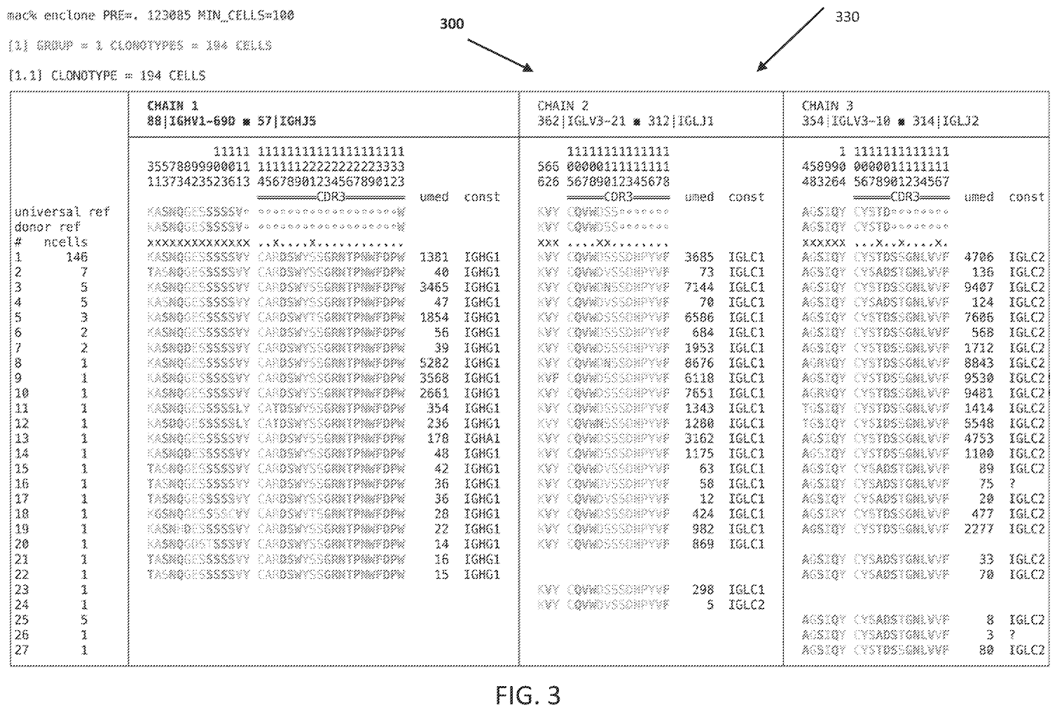

As discussed above, visualization 100 can also include a listing of one or more textual frames 130, in accordance with various embodiments. Frames 130 can include information about chains common to each member of the immune cell clonotype population. Frames 130 can include an amino acid or nucleotide sequence for the variable and constant regions of each subclonotype. Visualization 100 will generally output one or more frames 130. FIGS. 1, 2 and 5 illustrate two textual frames while FIG. 3 illustrates three textual frames.

Frames 130 can display many different types of information, but can also be readily configured via user instruction to display those many different types of information in virtually any combination.

Frames 130 can show positional information 134 for each member of the amino acid sequence. Frames 130 can include a listing of amino acid or nucleotide differences 140 between each subclonotype 124 of the clonotype population. An “x” 150 is shown in FIG. 1 at a column position where variation occurs within the clonotype. These “x” notations can comprise the raw evolutionary history of the clonotype, the positions containing information relevant to calculating an antibody phylogeny. Numbered columns 152 show the state of a particular amino acid. For example, reading vertically, the first column of the first chain shows a “20”, which can represent amino acid 20 in the first chain (where 0 is the start codon). The symbol [°] represent holes 154 in the recombined region where the reference does not make sense, specifically where it is too difficult to confidently identify where the reference sequence ends and where the junction region begins.

Amino acids can be colored in a fashion dependent on which detected codon represents a given amino acid. Moreover, synonymous changes can be displayed using different colors to display variability between subclonotypes with different nucleotide sequences at variable positions but identical amino acid sequences. A synonymous mutation is a change in the DNA sequence that codes for amino acids in a protein sequence, but does not change the encoded amino acid. Due to the redundancy of the genetic code (multiple codons code for the same amino acid), these changes usually occur in the third position of a codon. On frames 130, amino acids are displayed associated with a specific exact subclonotype if the displayed amino acid 160 differs from the universal reference sequence or the displayed amino acid 162 is also in the CDR provided (see “CDR3=CARRYFGVVADAFDIW” in grouping statement 114).

Frames 130 can show a comparison of at least one reference sequence 132 to a subclonotype 124. The at least one reference sequence can include a reference sequence listing selected from the group consisting of a universal reference sequence, a donor reference sequence, and combinations thereof. A universal reference is a sequence found in a public database and often the single sequence for a given genomic segment that is found in the reference sequence for the given species. A donor reference sequence is a modified version of this universal reference sequence that has mutations introduced, that are believed to have arisen in the germline sequence of the donor. The donor reference sequence is derived using data from the immune receptor dataset, where V segments (in various embodiments, also D and J segments) from multiple cells are used to impute shared mutations between different clonotypes, where the shared mutations represent the germline mutations found in a given V, D, or J gene of a donor. These mutations are found by observing mutations that are common to several different clonotypes sharing a given segment. FIG. 1, for example, displays both reference sequences, as does FIGS. 2, 3 and 5. Frames 130 can display germline changes as well, which are allelic variations distinct from variations caused by somatic hypermutation. For example, the notation “181.1.1” for chain 1 on FIG. 1 can mean that this V reference sequence is an alternate allele derived from the universal reference sequence (contig in the reference file) numbered 181, that is from donor 1 (hence “181.1”), and is the alternate allele 1 for that donor (hence “181.1.1”).

For each subclonotype, the textual frames 130 can provide chain-specific subclonotype information selected from the group consisting of V(D)J unique molecular identifier (UMI) count, V(D)J read count, constant region name, complementarity-determining region (CDR) sequence, constant sequence length, 5′UTR sequence length, differences from a universal reference constant region, differences from the 5′UTR sequence, base differences between subclonotypes, and combinations thereof. Referring to FIG. 1, for example, the provided chain-specific subclonotype information includes median UMI read count 144 for each exact clonotype and constant region name 146 associated with each chain in the given exact subclonotype. Median, maximum, mean, and similar summary statistics thereof can also be used in accordance with various embodiments to visualize and report the aforementioned features in addition to subclonotype. Those knowledgeable in the art recognize that there are many additional such features that could be reported such as percentage of a given set of features within a single cell and other user-provided annotations for a set of single cells such as manual annotation or description of information relevant to one or more subclonotypes, as specified in a variety of file formats.

Regarding UMI, for a given chain, a given cell contains a certain number of mRNA molecules representing that chain. Each of those that is reverse transcribed is tagged with a UMI, and the total number of UMIs that is found is thus a downward-biased estimate, for a given chain in a given cell, of the number of mRNA molecules that were present. For a given chain in a given exact subclonotype, is the median of the UMI counts for all the cells in the exact subclonotype (for the given chain). In accordance with various embodiments, it should be noted that, at times, some chains are missing from exact clonotypes. Take FIG. 1 for example, where subclonotype #3 is missing a second chain.

For more detail regarding customization of visualizations, in accordance with various embodiments, refer to the Additional Features section below for detailed discussion. It should be noted that the various parameters, variables, fields, values, filters, etc. discussed in detail herein are independent and interchangeable in any contemplated fashion or combination. Moreover, the various parameters, variables, fields, values, filters, etc. discussed in detail herein are applicable to any and all the various embodiments discussed or contemplated herein.

Referring to FIG. 2, another example visualization of identified clonotypes is provided, in accordance with various embodiments. This visualization 200 shares many similar characteristics to visualization 100 of FIG. 1. Of note is the subclonotypes listing frame 220. As discussed above, the visualization can also include a subclonotypes listing frame for an immune cell clonotype, in accordance with various embodiments. The subclonotypes can share identical V(D)J transcripts. Further, the subclonotypes listing frame 120 can include subclonotype information selected from the group consisting of gene expression, Hamming distance, antibody, and combinations thereof. The gene expression subclonotype information can be selected from the group consisting of median gene expression, maximum gene expression, mean gene expression, and combinations thereof. The gene expression subclonotype information can be reported as a UMI count. FIG. 2 illustrates various lead variables 270 not used in the example visualization 100 of FIG. 1. FIG. 2 illustrates lead variables for median gene expression 272 (reported as a UMI count), user selected gene 274 and user selected antibody 276. Reviewing command line 210, it is apparent that these lead variables sourced from a user input onto of optional parameters 214 next to dataset file path 212. This visualization (i.e., display) can be functional and helpful in the display of the measurement of antigen binding for clonotypes and subclonotypes.

Referring to FIG. 3, an example visualization of identified clonotypes is provided, in accordance with various embodiments. This visualization 300 of FIG. 3 shares many similar characteristics to visualizations 100/200 of FIGS. 1 and 2. Of note are textual frames 330 and the presence of a third chain not presented in first two example visualizations 100/200. Of note also are the missing chains of various exact subclonotypes, particularly subclonotypes 20 to 27. This visualization (i.e., display) can also be functional and helpful in the display of the measurement of antigen binding for clonotypes and subclonotypes.

Referring to FIG. 5, an example visualization of identified clonotypes is provided, in accordance with various embodiments. This visualization 500 of FIG. 5 shares many similar characteristics to the previously discussed visualizations. One note is the expanded use of lead variables 570. FIG. 5 illustrates lead variables for median gene expression 572 (reported as a UMI count), first user selected gene 574, second user selected gene 576, third user selected gene 578, and user selected antibody 580. Reviewing command line 510, it is apparent that these lead variables sourced from a user input onto of optional parameters 514 next to dataset file path 512.

In accordance with various embodiments, these visualizations (i.e., displays) can also be vertically expanded to display the same information at the per-barcode level in place of the per-subclonotype level. In accordance with various embodiments, these visualizations can be also be customized to group cells based on sample-level, clonotype-level, or barcode-level information (e.g., how many cells in a subclonotype are from a given time point or a given donor, etc.).

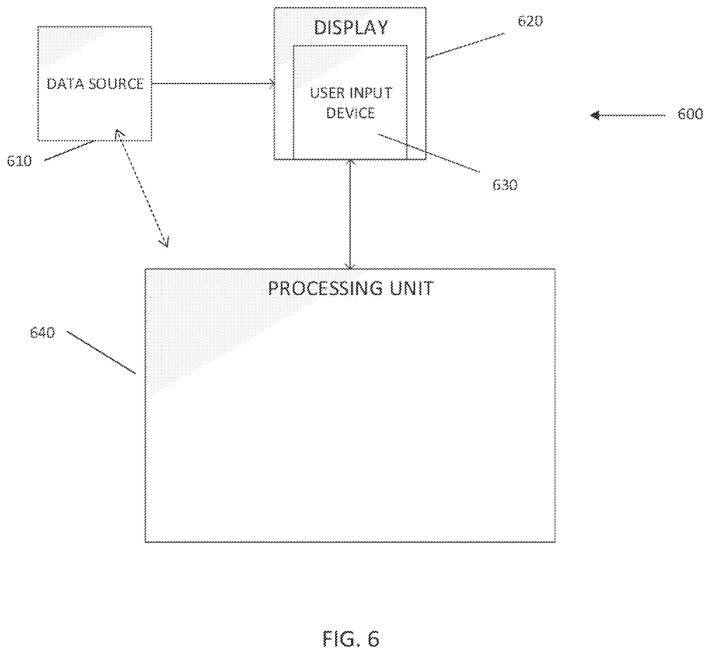

In accordance with various embodiments, FIG. 6 illustrates an interactive visualization system 600. System 600 can comprise a data source 610, a display 620, a user input device 630 and a processor 640. While user input device 630 is shown as part of display 620, it should be understood that these components also can be independent.

Note that all previous discussion of additional features, particularly with regard to the preceding described methods and graphical user interfaces, in accordance with various embodiments, are applicable to the features of the various system embodiments described and contemplated herein.

In accordance with various embodiments, the data source 610 can be configured to obtain a B cell receptor data set. Data source can be configured to obtain an immune cell sequence dataset from a sample, the dataset including a plurality of immune receptor sequences each comprised of a heavy chain region sequence and a light chain region sequence, wherein each variable domain region sequence is associated with an individual immune cell in the sample. User input device 630 can be configured to receive a user selected parameter under which to analyze the data set.

In accordance with various embodiments, the data source 610 can be configured to obtain a T cell receptor data set. Data source can be configured to obtain an immune cell sequence dataset from a sample, the dataset including a plurality of variable domain region sequences each comprised of an alpha chain sequence and/or a beta chain sequence and/or a gamma chain sequence and/or a delta chain sequence, wherein each variable domain region sequence is associated with an individual immune cell in the sample. User input device 630 can be configured to receive a user selected parameter under which to analyze the data set.

Processor 640 can be configured to identifying a clonotype group in the data set using the parameter, identify subclonotypes within the clonotype group, wherein each identified subclonotype comprises cells having identical V(D)J transcripts, and process the data to define a visualization model that can display a compressed view of the identified clonotype group.

Display 620 can be configured to render a visualization of said data set according to said visualization model, wherein the visualization displays the clonotype group by identified subclonotype.

In accordance with various embodiments, the parameter can be a first parameter, the visualization model can be a first visualization model, and the visualization can be a first visualization. Accordingly, the user input device 630 can be further configured to receive a second parameter under which to analyze the data set. Processor 640 can be further configured to re-identify a clonotype group in the data set using the second parameter, re-identify subclonotypes within the clonotype group, wherein each identified subclonotype comprises cells having identical V(D)J transcripts, and re-process the data to define a second visualization model that can display a modified compressed view of the identified clonotype group. Display 620 can be further configured to re-render a second visualization of said data set according to said second visualization model, wherein the second visualization displays a modified version of the clonotype group by identified subclonotype.

In accordance with various embodiments, the visualization can display a comparison of at least one reference sequence to a subclonotype. The at least one reference sequence can include a reference sequence listing selected from the group consisting of a universal reference sequence or user-supplied reference, a donor reference sequence, and combinations thereof.

In accordance with various embodiments, the visualization can display a listing of amino acid differences between each subclonotype of the clonotype population. In accordance with various embodiments, the visualization can display subclonotype information selected from the group consisting of gene expression, Hamming distance, antibody, and combinations thereof. The gene expression subclonotype information can be selected from the group consisting of median gene expression, maximum gene expression, mean gene expression, and combinations thereof. The gene expression subclonotype information can be reported as a UMI count.

In accordance with various embodiments, for each subclonotype, the visualization can display chain-specific subclonotype information selected from the group consisting of V(D)J UMI count, V(D)J read count, constant region name, complementarity-determining region (CDR) sequence, constant sequence length, 5′UTR sequence length, differences from a universal reference constant region, differences from the 5′UTR sequence, base differences between subclonotypes, and combinations thereof. Median, maximum, mean, and similar summary statistics thereof can also be used in accordance with various embodiments to visualize and report the aforementioned features in addition to subclonotype. Those knowledgeable in the art recognize that there are many additional such features that could be reported such as percentage of a given set of features within a single cell and other user-provided annotations for a set of single cells such as manual annotation or description of information relevant to one or more subclonotypes, as specified in a variety of file formats.

In accordance with various embodiments, processor 640 of system 600 of FIG. 6 can be communicatively connected to data source 610 (see dotted line in FIG. 6), display 620, and/or user input device 630. In various embodiments, processor 640 can include various engines configured to carry out the functionality of processor 640. It should be appreciated that each component (e.g., engine, module, unit, etc.) depicted as part of system 600 (and described herein) can be implemented as hardware, firmware, software, or any combination thereof.

In various embodiments, processor 640 can be implemented as an integrated instrument system assembly with any of data source 610, display 620, and user input device 630. That is, any combination of processor 640, data source 610, display 620, and user input device 630 can be housed in the same housing assembly and communicate via conventional device/component connection means (e.g. serial bus, optical cabling, electrical cabling, etc.).

In various embodiments, processor 640 can be implemented as a standalone computing device (as shown in FIG. 6) that can be communicatively connected to the data source 610 (and likewise display 620 and user input device 630) via an optical, serial port, network or modem connection. For example, the processor 640 can be connected via a LAN or WAN connection that allows for the transmission of data to and from the data source 610, and likewise display 620 and user input device 630.

In various embodiments, the functions of processor 640 can be implemented on a distributed network of shared computer processing resources (such as a cloud computing network) that is communicatively connected to the data source 610 via a WAN (or equivalent) connection. For example, the functionalities of processor 640 can be divided up to be implemented in one or more computing nodes on a cloud processing service such as AMAZON WEB SERVICES™.

Within the processor 640, any internal engines can be implemented as separate engines or a single multi-functional engine. As such, FIG. 6 simply provides one example implementation of a system in accordance with various embodiments, and should be not be read to limit the interchangeability, interoperability and/or functionality of all the components therein.

In accordance with various embodiments, various features can be provided to supplement the various embodiments provided herein.

As stated above, visualization of identified clonotypes can source from single cell datasets. Mechanisms for calling specific datasets can originate from various sources that include, for example, entering the data source path directly on the command line (see FIGS. 1 and 2 for examples) or via one or more supplementary metadata files.

When entering the data source path directly on the command line, a common entry simply points at specific input files as shown by the portion circled on FIGS. 1 and 2. For a more complicated syntax, punctuation can be used such as, for example, commas, colons and semicolons that can act as delimiters. Commas can be used, for example, between datasets from the same sample. Colons can be used, for example, between datasets from the same donor. Semicolons can be used to separate donors. Using this input system, each dataset can assigned an abbreviated name, which can be everything after the final slash in the directory name (e.g. “enclone_data” in FIGS. 1 and 2). The entire name of a dataset can be used, for example, when there is no slash. Moreover, samples and donors can be assigned numerical identifiers starting at one. Using this system, a base example of input data from two libraries from the same sample can be exemplified (e.g., TCR=p1,p2), an example of the same input data plus another from a different sample from the same donor can be exemplified (e.g., TCR=p1,p2:q), and example of input data of one library from each of two donors can be exemplified (e.g., TCR=“a;b”). Likewise, matching gene expression and/or feature barcode data may also be supplied using an argument “GEX= . . . ” (see command line of FIG. 2, for example).

To specify a metadata file, as opposed to entering a data source directly on the command line, a user can implement a specific command line argument calling a metadata file (e.g, META=filename). The file can be in a CSV format (comma-separated values) or tab-separated/character-delimited data format. In addition to the metadata file call, other fields can be used to provide further parameters. For example, a field such as “tcr” or “bcr” can be used to provide a path to the dataset, wherein the full file name can be used or an abbreviated name for the data set can be used, generally with a designation that an abbreviated name is being used (e.g., “abbr”). Further, a field such as “gex” can be used to provide a path to the gene expression dataset, which may include of consist of a function-based (FB) dataset. Further fields such as, for example, “sample” or “donor” can be used to provide a name, or abbreviated name of a sample or donor respectively. To specify information about individual cell barcodes, a user can implement a specific command line argument calling barcode-level data from a file (e.g., BC=filename). The file can be in a CSV format or tab-separated/character-delimited data format. The file can include a barcode field and any other fields of interest, such as origin, donor, tag, or color fields. Origin and donor fields may allow a particular origin and donor to be associated with a given barcode for use in, for instance, genetic demultiplexing. A tag field may allow a particular tag to be associated with a given barcode for use in, for instance, tag demultiplexing.

When specifying a CDR sequence in the command line, the sequence can be input various ways. For example, one could require an exact sequence (e.g., CDR3=CARPKSDYIIDAFDIW), at least one of multiple sequences (e.g., CDR3=“CARPKSDYIIDAFDIW|CQVWDSSSDHPYVF”), or a snippet of a sequence inside the CDR sequence (e.g., “.*DYIID.*”), where quotations are used when non-letter characters are provided (e.g., “.”, “*”, “|”).

In accordance with various embodiments, the output visualization can be customized in a variety of ways to provide the user desired targeted output information and augment the output. Customization can be based on, for example, cell count, unique-molecular-identifier (UMI) count, chain count, CDR (e.g., CRD3) patterns, V(D)J segment specification, subclonotype count, VJ segment specification, cross-data set cell comparisons, universal reference comparisons, deletion specificity, antigen specificity, or other clonotype/subclonotype/barcode-specific information provided as metadata in parallel to the application.

For cell count customization, fields can be used to show clonotypes having at least n cells (e.g., MIN_CELLS=n), show clonotypes having at most n cells (e.g., MAX_CELLS=n), or show clonotypes having exactly n cells (e.g., CELLS=n). For UMI count customization, fields can be used to show clonotypes having ≳n UMIs on some chain on some cell (e.g., MIN-UMIS=n).

For chain count customization, fields can be used to show clonotypes having at least n chains (e.g., MIN_CHAINS=n), show clonotypes having at most n chains (e.g., MAX_CHAINS=n), show clonotypes having exactly n chains (e.g., CHAINS=n). For CDR patterns, fields can be used to show clonotypes having a CDR3 amino acid sequence that matches a given pattern, from beginning to end (e.g., CDR3=<pattern>).

For V(D)J segment specification, fields can be used to show clonotypes using one of the given VDJ segment names (double quotes can be used if n>1) (e.g., “SEG=s_1| |s_n”), or show show clonotypes using one of the given VDJ segment numbers (double quotes only needed if n>1) (e.g., “SEGN=s_1| . . . |s_n”).

For subclonotype count specification, fields can be used to show clonotypes having at least n exact subclonotypes (e.g., MIN_EXACTS=n). For VJ segment specification, fields can be used to show clonotypes using exactly the given V . . . J sequence (string in alphabet ACGT) (e.g., VJ=seq).

For cross-data set cell comparisons, fields can be used to show clonotypes containing cells from at least n datasets (e.g., MIN_DATASETS=n). For universal reference comparisons, fields can be used to show clonotypes having a difference in constant region with the universal reference (e.g., CDIFF). For deletion specificity, fields can be used to show clonotypes exhibiting a deletion (e.g., DEL).

In accordance with various embodiments, the output visualization can be customized with a variety of filtering options to provide the user desired targeted output information and augment the output. These filtering options could include turning on a filter or turning off a filter.

In accordance with various embodiments, the output visualization can be customized with a variety of options to suppress or display additional output. An example of an output option is an export filter. If one specifies that export of the donor-derived reference, FASTA nucleotide sequence of an exact subclonotype, FASTA amino acid sequence of an exact subclonotype, or of a selection of any or a subset of the fields generated by analysis should be performed, then these features can be displayed and simultaneously written to a user-specified file in the appropriate format.

An example of a filtering option is a cross-filter. If one specifies that two or more libraries arose from the same sample (i.e., from the same tube of cells), then the default behavior of the various embodiments herein, can be to “cross filter” so as to remove expanded exact subclonotypes that are present in one library but not another, in a fashion that would be highly improbable, assuming random draws of cells from the tube. Such observed behavior can be understood to arise when a plasma or plasmablast cell breaks up during or after pipetting from the tube, and the resulting fragments seed can yielding ‘fake’ cells. This filter, presumably defaulted to being on during sample analysis of subclonotype identification, can also be turned off per user input. It is understood that the reverse is also contemplated.

Another example of a filtering option relates to a filter that, by default in various embodiments, removes exact subclonotypes that by virtue of their relationship to other exact subclonotypes, appear to arise from background mRNA or a phenotypically similar phenomenon. This filter, presumably defaulted to being on during sample analysis of subclonotype identification, can also be turned off per user input. It is understood that the reverse is also contemplated.

Another example of a filtering option relates to a filter that, by default in various embodiments, filters out exact subclonotypes having a base in V(D)J sequence that looks like it might be wrong. A Phred quality score (Q score) is a measure of the quality of the identification of the nucleobases generated by automated DNA sequencing. Various methods, in accordance with various embodiments herein, can find bases which are not Q60 for a barcode, not Q40 for two barcodes, are not supported by other exact subclonotypes, are variant within the clonotype, and which disagree with the donor reference. This filter, presumably defaulted to being on during sample analysis of subclonotype identification, can also be turned off per user input. It is understood that the reverse is also contemplated.

Another example of a filtering option relates to a filter that, by default in various embodiments, filters out chains from clonotypes that are weak and appear to be artifacts, perhaps arising from, for example, a stray mRNA molecule. This filter, presumably defaulted to being on during sample analysis of subclonotype identification, can also be turned off per user input. It is understood that the reverse is also contemplated.

Another example of a filter relates to a filter that, by default in various embodiments, identifies and filters out cells with low credibility, or barcode-associated rearrangements that artificially inflate the size of a given clonotype. This filter operates by using V(D)J sequence data in addition to one or more modes of data for the same cells. This filter is comprised of multiple steps, each of which can be run independently or in combinations with any of the other steps. These steps may include: (1) removal of V(D)J cells and chains that are not present in the second dataset (for example, remove of V(D)J cells if those cells are not also found in the orthogonal gene expression dataset); (2) for a clonotype of n cells, determining for each cell in the clonotype, the n nearest neighbors in an appropriate dimensional reduction or using a sensible distance metric to find these neighbors' gene expression or other dataset; and (3) calculating the credibility of a cell, where credibility is the percent of those nearest neighbors meeting at least one or more of the following criteria: (a) where the nearest neighbors are also V(D)J-called cells, (b) where the nearest neighbors are immune cells, e.g., B or T cells, identified by supervised analysis, (c) where the nearest neighbors are immune cells, e.g., B or T cells identified by supervised analysis, and (d) where the nearest neighbors are a non-B or non-T cell or a cell that should not otherwise express a B or T cell receptor. This filter can also use the nearest neighbor graph from various clustering algorithms (e.g. the Leiden or Louvain algorithms, and other commonly known algorithms) to calculate credibility of cells by: (1) measuring the geodesic distance between a cell and its n nearest neighbors in the graph; and (2) determining which of those nearest neighbors meet the comparison criteria listed above. This filter, presumably defaulted to being on for identifying and filtering out cells with low credibility, or barcode-associated rearrangements that artificially inflate the size of a given clonotype, can also be turned off per user input. It is understood that the reverse is also contemplated.

Another example of a filtering option relates to a filter that, by default in various embodiments, filters out onesie clonotypes (a clonotype or exact subclonotype having exactly one chain) having a single exact subclonotype, and that are light chain or TRA gene, and whose number of cells is less than, for example, 0.1% of the total number of cells. This filter, presumably defaulted to being on during sample analysis of subclonotype identification, can also be turned off per user input. It is understood that the reverse is also contemplated.

Another example of a filtering option relates to a filter that, by default in various embodiments, finds a foursie exact subclonotype that contains a twosie exact subclonotype having at least ten cells, it kills the foursie exact subclonotype, no matter how many cells it has. The foursies that are killed are believed to be rare odd artifacts arising from repeated cell doublets or, for example, GEMs (Gel bead-in-EMulsion) that contain two cells and multiple gel beads. This filter, presumably defaulted to being on during sample analysis of subclonotype identification, can also be turned off per user input. It is understood that the reverse is also contemplated.

Another example of a filtering option relates to a filter that, by default in various embodiments, filters out rare artifacts arising from contamination of oligos on gel beads. This filter, presumably defaulted to being on during sample analysis of subclonotype identification, can also be turned off per user input. It is understood that the reverse is also contemplated.

Another example of a filtering option relates to a filter that, by default in various embodiments, labels an exact subclonotype as improper if it does not have one chain of each type. This filtering option causes all improper exact subclonotypes to be retained, although they may be removed by other filters.

Another example of a filter relates to a filter that, by default in various embodiments, can be used to select exact subclonotypes within a specified range of generation probability, where the generation probability is calculated by calculating the likelihood of a specific rearrangement being generated relative to rearrangements generated in silico. In some embodiments, the generation probability is conditioned on the V gene used in the observed rearrangement. In some embodiments, spurious subclonotypes that may have been identified by de novo assembly or that arose due to chemistry errors can be removed by application of this filter in combination with other filters described. This filter, presumably defaulted to being on during sample analysis of exact subclonotype identification, can also be turned off per user input. It is understood that the reverse is also contemplated

Yet another example of a filtering option relates to a filter that, by default in various embodiments, deletes any exact subclonotype having less than n chains. Such a filter can be used to “purify” a clonotype so as to display only exact subclonotypes having all their chains. Similarly, another example of a filtering option relates to a filter that, by default in various embodiments, deletes any exact subclonotype having less than n cells. Such a filter can be used for a very large and complex expanded clonotype, for which it may be desired to see a simplified view.

In accordance with various embodiments, the output visualization can be customized with a variety of lead variable and per-chain variable options to provide the user desired targeted output information and augment the output. Lead variable options (LVARS) can be formatted to appear once for each clonotype and, as shown in FIG. 2, can be provided along the left, side, with one entry for each subclonotype row. FIG. 2, shows LVARS as “gex-med”, “IGHV2-5_g” and “CD4_a”. LVARS can be specified in the example format LVARS=x1, . . . xn. The variable x can be related to datasets, donors, cells, gene expression UMI count, Hamming distance, gene expression data, and feature barcode data.

Regarding datasets and donors, a lead variable referencing donor or dataset identifiers can be used. Regarding cells, lead variables can be used that (a) provide an n number of cells or (b) provide an n number of cells associated to a given name, which can be, for example, a dataset short name, a sample short name, a donor short name, and so on. Regarding gene expression UMI count, lead variables can be use that request a median gene expression UMI count or a max gene expression UMI count. Regarding Hamming distance, lead variables can be used that request a Hamming distance of a V . . . J DNA sequence to its nearest neighbor and a V . . . J DNA sequence to its farthest neighbor. Another example using Hamming distance involves grouping all exact subclonotypes according to the Hamming distance of their V . . . J sequences. More specifically, those within distance d are defined to be in the same group, and this is extended transitively. A group identifier 1, 2, etc. can be provided, the order of which can be arbitrary. Hamming distance comparisons can be usefully applied in various situations such as, for example, cases where all exact subclonotypes have a complete set of chains. Regarding feature barcode data, lead variables can be used that (a) assume that feature barcode data has been provided, (b) look for a feature line that starts with the given name, and (c) then has a tab—the report out being in the form of mean UMI count value. Regarding gene expression data, lead variables can be used that (a) assume that gene expression data has been provided, and (b) look for a feature line that starts with the given name in the second tab delimited column—the report out being in the form of mean UMI count value. In accordance with various embodiments, default LVARS can be, for example, dataset identifiers and n number of cells.

Regarding per-chain variable options (CVARS), these options define per-chain variables, which correspond to columns that appear once for each chain in each clonotype, and have one entry for each exact subclonotype. CVARS can be specified in the example format CVARS=x1, . . . xn. The variable x can be related to varying bases in chain (e.g., bases at positions in chain that vary across the clonotype), UMI counts, read counts (median VDJ read count for each exact subclonotype), constant region name, a measure of CDR3 complexity, CDR3 DNA sequence, various sequence lengths and differences, optional notes (optional note if there is an insertion, omitted if empty), and base differences (number of base differences within V . . . J with exact subclonotype n).

Regarding UMI counts, CVARS can be used that request median VDJ UMI count for each exact subclonotype, max VDJ UMI count for each exact subclonotype, or total VDJ UMI count for each exact subclonotype. Regarding various sequence lengths and differences, CVARS can be used that requests length of observed constant sequence (usually truncated at primer start) or length of observed 5′-UTR sequence. CVARS can be used that requests differences versus a universal reference constant region, which can be shown in the abbreviated form e.g. 22T (ref changed to T at base 22) or 22T+10 (same but contig has 10 additional bases beyond end of ref C region). In accordance with various embodiments, default CVARS can be, for example, median VDJ UMI count for each exact subclonotype, constant region name and optional notes (optional note if there is an insertion, omitted if empty).

In accordance with various embodiments, the output visualization can be customized with a variety of amino acid related variables (AMINO) to provide the user desired targeted output information and augment the output. There is a complex per-chain column that can be to the left of other per-chain columns, and can be specified according to the entry AMIN0=x1, . . . , xn, which can result in the display of amino acid columns for the given categories, in one combined ordered group. The categories x can be one or more of CDR3 sequence, positions in chain that vary across the clonotype, positions in chain that differ consistently from the donor reference, positions in chain where the donor reference differs from the universal reference, and positions in chain where the donor reference differs non-synonymously from the universal reference.

In accordance with various embodiments, the output visualization can be customized with a variety of display options for controlling clonotype display, which can provide the user desired targeted output information and augment the output. One option is a per barcode expansion, where each exact clonotype line is expanded, showing one line per barcode, for each such line, displaying the barcode name, the number of UMIs assigned, and the gene expression UMI count, if applicable, under gex_med (see above). Another option is a barcode list, whereby a list of all barcodes of the cells in each clonotype is printed in a single line near the top of the printout for a given clonotype. Another option is to print the V . . . J sequence for each chain in the first exact subclonotype, near the top of the printout for a given clonotype. Another option is to print the full sequence for each chain in the first exact subclonotype, near the top of the printout for a given clonotype. An option for controlling clonotype grouping is to group clonotypes by perfect identity of CDR3 amino acid sequence of IGH or TRB, or group by minimum number of clonotypes in group to print.

In accordance with various embodiments, the output visualization can be customized with a variety of options handling insertions and deletions, which can provide the user desired targeted output information and augment the output. The various embodiments described herein can be configured to recognize and display a single insertion or deletion in a contig relative to the reference. Such recognition and display can be subject to standards, such as the indel length being divisible by three, being relatively short, and occurring within the V segment, but not too close to its right end. These indels can be germline, however most such events are already captured in a reference sequence. Deletions can be displayed using hyphens (-). If the var option for CVARS (see above) is used, the hyphens can be displayed in base space, where they are initially observed. For the AMINO option (see above), the deletion can be first shifted by up to two bases, so that the deletion starts at a base position that is divisible by three. The deleted amino acids can be shown as hyphens. Insertions can be shown in amino acid space, in a special per-chain column that appears if there is an insertion. Colored amino acids are shown for the insertion, and the position of the insertion can be shown. The position is the position of the amino acid after which the insertion appears, where the first amino acid (start codon) is numbered 0.

In accordance with various embodiments, the output visualization can be customized with a variety of options to provide the user desired output information regarding a phylogenetic analysis. In various embodiments, the output visualization may display a phylogenetic tree derived from a phylogenetic analysis (for example, from a Newick file or a Clustal file). In various embodiments, the distance between any two subclonotypes may be defined as approximately equal to a Levenshtein distance between them. A root “virtual” exact subclonotype may be added, which may be approximately equal to a donor reference away from the recombination region. The root subclonotype may be undefined within that region (for example, the root subclonotype may be a germline-reverted exact clonotype without the junction). The distance from the root subclonotype to any actual exact subclonotype may be approximately equal to a Levenshtein distance away from the region of recombination. A phylogenetic tree may be created from the set of Levenshtein distance data. For example, the phylogenetic tree may be created from the set of Levenshtein distance data using a neighbor joining algorithm. Negative distances may be changed to zero. In some embodiments, the output visualization contains the phylogenetic tree in a plain text format. In some embodiments, output visualization contains the phylogenetic tree in a Newick format. In some embodiments, the output visualization contains the phylogenetic tree in a Clustal format. The Clustal format may comprise a Clustal alignment for each clonotype (for example, using either nucleic acid bases or amino acids), with one sequence for each exact subclonotype. The sequence may comprise a concatenation of per-chain sequences, with an appropriate number of gap (-) characters shown if a chain is missing.

In accordance with various embodiments, the output visualization can be customized with a variety of options to provide the user desired output information regarding amino acid or clonotype consensus sequences. In various embodiments, the output visualization can be customized to provide the user with a consensus for CDR3 across a clonotype. The output visualization may be customized to display an “X”, or other symbol, demarking each variant residue within the clonotype. The output visualization may be customized to show a property symbol whenever two different amino acids are observed. For example, the output visualization may be customized to show a “B,” “Z,” “J”, “−,” “+,” “Ψ,” “Π,” “Ω,” “Φ,” or “ζ” whenever an asparagine or aspartic acid, glutamine or glutamic acid, leucine or isoleucine, negatively charged, positively charged, aliphatic, small, aromatic, hydrophobic, or hydrophilic amino acid, respectively, are observed.

In accordance with various embodiments, the output visualization can be customized with a variety of options to provide the user desired output information regarding the count and/or location of user-specified amino acid motifs.

In accordance with various embodiments, the output visualization can be customized with a variety of options to provide the user desired output information regarding CDR and/or FWR sequences. In some embodiments, the CDR and/or FWR sequences are displayed in in a North format. In some embodiments, the CDR and/or FWR sequences are displayed in a specified extension length format.

In accordance with various embodiments, the output visualization can be customized with a variety of options to provide the user desired output information in any coloring scheme. In some embodiments, the output visualization can color amino acids by codon. For example, different codons coding for the same amino acid may be colored differently. For example, the GCT codon may be colored light blue, the GCC codon may be colored pink, the GCA codon may be colored dark blue, and the GCG codon may be colored green. Each of these codons may code for alanine. Other coloring schemes may be used for alanine or for any other amino acid. In some embodiments, the output visualization can color amino acids by their properties. For example, aliphatic amino acids (such as alanine, glutamine, isoleucine, leucine, proline, and/or valine) can be colored a first color, such as light blue. Aromatic amino acids (such as phenylalanine, tryptophan, and/or tyrosine) can be colored a second color, such as red. Acidic amino acids (such as aspartic acid and/or glutamic acid) can be colored a third color, such as orange. Basic amino acids (such as arginine, histidine, and/or lysine) may be colored a fourth color, such as dark blue. Hydroxylic amino acids (such as serine and/or threonine) may be colored a fifth color, such as pink. Sulfurous amino acids (such as cysteine and/or methionine) may be colored a sixth color, such as green. Amidic amino acids (such as asparagine and/or glutamine) may be colored a seventh color, such as yellow.

In accordance with various embodiments, the output visualization can be customized with a variety of options to provide the user desired output information about a variety of features or measurements. In some embodiments, the desired output information comprises user-specified combinations of features or measurements that select or filter clonotypes. The output visualization may show only clonotypes having at least, at most, or exactly some number of cells. The output visualization may show only clonotypes having at least, at most, or exactly some number of chains. The output visualization may show only clonotypes having a CDR3 amino acid sequence that matches some pattern. The output visualization may show only clonotypes using a given reference segment name or segment number. The output visualization may show only clonotypes having at least, at most, or exactly some number of subclonotypes. The output visualization may show only clonotypes containing cells from at least, at most, or exactly some number of datasets. The output visualization may show only clonotypes having a difference in constant region with a universal reference. The output visualization may show only clonotypes exhibiting one or more deletions. The output visualization may show only clonotypes annotated as having some iNKT or MAIT evidence. The output visualization may show only clonotypes satisfying any combination of any of the preceding.

Various user commands may provide commands to customize the output visualization. Table 1 shows examples of such commands.

TABLE 1
Commands for customizing the output visualization

| Variable | Brief description |
|---|---|
| (from BC or META/bc) | user defined variable |
| (from INFO) | user defined variable |
| <feature> | count for a gene expression or antibody feature |
| <feature>_% | percent of total expression for a particular gene |
| <feature>_max | maximum count for a feature |
| <feature>_mean | mean count for a feature (same with μ for mean) |
| <feature>_min | minimum count for a feature |
| <feature>_sum | sum of counts for a feature (same with Σ for sum) |
| <feature>_Σ | sum of counts for a feature (same with sum for Σ) |
| <feature>_μ | mean count for a feature (same with mean for μ) |
| <dataset>_barcode | barcode from the given dataset (or null) |
| <dataset>_barcodes | barcodes from the given dataset |
| aa % | amino acid identity with donor reference |
| barcode | barcode of the cell |
| barcodes | barcodes for the exact subclonotype |
| (from BC or META/bc) | user defined variable |
| (from INFO) | user defined variable |
| <feature> | count for a gene expression or antibody feature |
| <feature>_% | percent of total expression for a particular gene |
| <feature>_max | maximum count for a feature |
| <feature>_mean | mean count for a feature (same with μ for mean) |
| <feature>_min | minimum count for a feature |
| <feature>_sum | sum of counts for a feature (same with Σ for sum) |
| <feature>_Σ | sum of counts for a feature (same with sum for Σ) |
| <feature>_μ | mean count for a feature (same with mean for μ) |
| <dataset>_barcode | barcode from the given dataset (or null) |
| <dataset>_barcodes | barcodes from the given dataset |
| aa % | amino acid identity with donor reference |
| barcode | barcode of the cell |
| barcodes | barcodes for the exact subclonotype |
| cdiff | differences of const region with universal reference |
| cdr*_aa | CDR* amino acid sequence |
| cdr*_aa_L_R_ext | CDR* region with specified extension length |
| cdr*_aa_north | North version of CDR* amino acid sequence |
| cdr*_aa_ref | CDR* amino acid sequence for universal reference |
| cdr*_dna | CDR* nucleotide sequence |
| cdr*_dna_ref | CDR* nucleotide sequence for universal reference |
| cdr*_len | length of CDR* amino acid sequence |
| cdr3_aa_conp | CDR3 amino acid consensus, symbols at variants |
| cdr3_aa_conx | CDR3 amino acid clonotype consensus, Xs at variants |
| cdr3_start | nucleotide start of CDR3 sequence on full sequence |
| clen | length of observed constant region |
| clonotype_id | identifier of clonotype within clonotype group |
| clonotype_ncells | number of cells in the clonotype |
| comp | CDR3 complexity number |
| const | constant region name |
| const_id | numerical identifier of constant region (or null) |
| count_* | count amino acid motifs |
| cred | credibility assessed using GEX data |
| _donor | distance from donor reference |
| d_frame | reading frame of D segment (0, 1, 2 or null) |
| d_id | D region id |
| d_name | D region name |
| d_start | start of D on full nucleotide sequence (or null) |
| d_univ | distance from universal reference |
| datasets | dataset names |
| dna % | nucleotide identity with donor reference |
| donors | donor names |
| dref | nucleotide distance to donor reference |
| dref_aa | amino acid distance to donor reference |
| edit | edit to get from reference CDR3 |
| exact_subclonotype_id | identifier of exact subclonotype |
| filter | name of filter that would be applied (if filters off) |
| fwr*_aa | FWR* amino acid sequence |
| fwr*_aa_ref | FWR* amino acid seq for universal reference |
| fwr*_dna | FWR* nucleotide sequence |
| fWr*_dna_ref | FWR* nucleotide seq for universal reference |
| fwr*_len | length of FWR* amino acid sequence |
| g<d> | exact subclonotype group, by Hamming distance |
| gex | number of GEX UMIs |
| gex_max | maximum number of GEX UMIs across exact subclonotype |
| gex_mean | mean of GEX UMIs across exact subclonotype (=gex_μ) |
| gex_min | minimum number of GEX UMIs across exact subclonotype |
| gex_sum | sum of GEX UMIs across exact subclonotype (=gex_Σ) |
| gex_Σ | sum of GEX UMIs across exact subclonotype (=gex_sum) |
| gex_μ | mean of GEX UMIs across exact subclonotype (=gex_mean) |
| group_id | identifier of clonotype group |
| group_ncells | number of cells in clonotype group |
| inkt | evidence for iNKT cell |
| j_id | J region id |
| j_name | J region name |
| mait | evidence for MAIT cell |
| n | number of cells |
| n_<name> | number of cells associated to the given name |
| n_gex | number of cells seen by GEX pipeline |
| nchains | number of chains in the clonotype |
| ndiff<n>vj | number of base differences with exact subclonotype n |
| near | Hamming distance to nearest neighbor |
| notes | notes for exact subclonotype |
| origins | origin names |
| q<n>_{—} | read quality scores at position n |
| r | number of reads supporting chain |
| r_max | maximum chain read count across exact subclonotype |
| r_mean | mean chain reads across exact subclonotype (=r_mean) |
| r_min | minimum chain read count across exact subclonotype |
| r_sum | sum of chain read counts across exact subclonotype (=r_Σ) |
| r_Σ | sum of chain reads across exact subclonotype (=r_sum) |
| r_μ | mean chain read count across exact subclonotype (=r_μ) |
| seq | full nucleotide sequence of exact subclonotype |
| share_indices_aa | shared amino acid positions |
| share_indices_dna | shared nucleotide positions |
| u | number of UMIs supporting chain |
| u_max | maximum chain UMIs across exact subclonotype |
| u_mean | mean chain UMIs across exact subclonotype (=u_μ) |
| u_min | minimum chain UMIs across exact subclonotype |
| u_sum | sum of chain UMIs for exact subclonotype (=u_Σ) |
| u_Σ | sum of chain UMIs across exact subclonotype (=u_sum) |
| u_μ | mean chain UMIs for exact subclonotype (=u_mean) |
| udiff | differences of 5′-UTR region with universal reference |
| ulen | length of observed 5′-UTR sequence |
| utr_id | numerical identifier of 5′-UTR region (or null) |
| utr_name | name 5′-UTR region (or null) |
| v_id | V region id |
| v_name | V region name |
| v_start | start of V on full nucleotide sequence |
| var | bases at position in chain that vary across the clonotype |
| var_aa | variant residue indices in clonotype (including |
| | synonymous) |
| var_indices_aa | variable amino acid positions |
| var_indices_dna | variable nucleotide positions |
| vj_aa | amino acid sequence of V . . . J |
| vj_aa_nl | amino acid sequence of V . . . J, excluding leader |
| vj_seq | nucleotide sequence of V . . . J |
| vj_seq_nl | nucleotide sequence of V . . . J, excluding leader |
| vjlen | length in bases of V . . . J |

### Computer-Implemented System

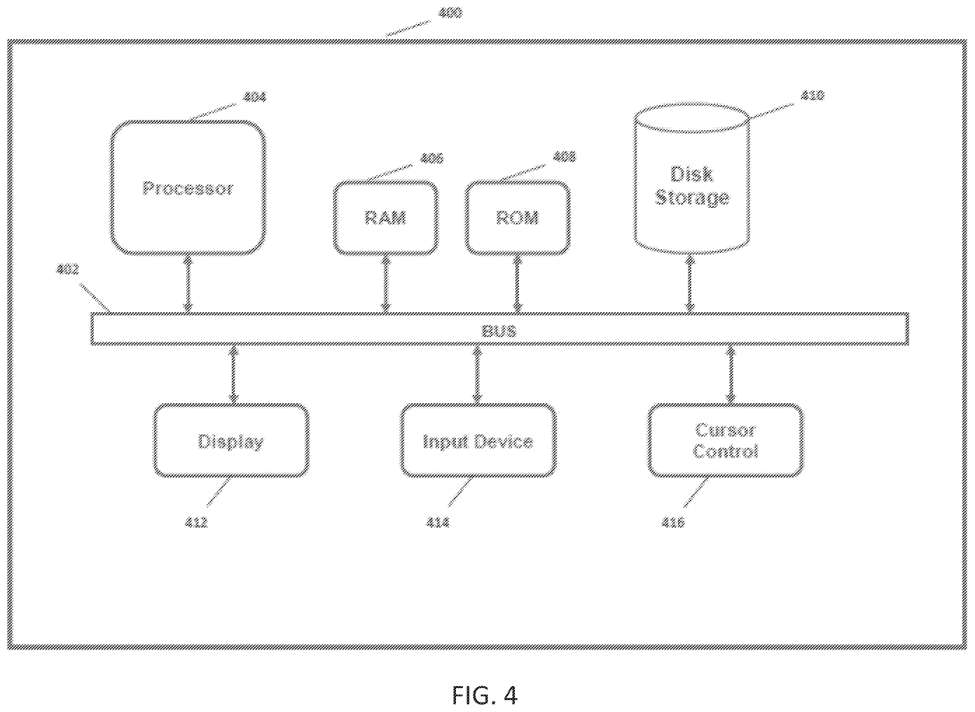

FIG. 4 is a block diagram that illustrates a computer system 400, upon which embodiments of the present teachings may be implemented. In various embodiments of the present teachings, computer system 400 can include a bus 402 or other communication mechanism for communicating information, and a processor 404 coupled with bus 402 for processing information. In various embodiments, computer system 400 can also include a memory, which can be a random access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a trackball or cursor direction keys for communicating direction information and command selections to processor 404 and for controlling cursor movement on display 412. This input device 414 typically has two degrees of freedom in two axes, a first axis (i.e., x) and a second axis (i.e., y), that allows the device to specify positions in a plane. However, it should be understood that input devices 414 allowing for 3 dimensional (x, y and z) cursor movement are also contemplated herein.

Consistent with certain implementations of the present teachings, results can be provided by computer system 400 in response to processor 404 executing one or more sequences of one or more instructions contained in memory 406. Such instructions can be read into memory 406 from another computer-readable medium or computer-readable storage medium, such as storage device 410. Execution of the sequences of instructions contained in memory 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as memory 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Rust, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400 of Appendix D, whereby processor 404 would execute the analyses and determinations provided by these engines, subject to instructions provided by any one of, or a combination of, memory components 406/4008/410 and user input provided via input device 414.

### Digital Processing Device

In various embodiments, the systems and methods described herein can include a digital processing device, or use of the same. In various embodiments, the digital processing device can includes one or more hardware central processing units (CPUs) or general-purpose graphics processing units (GPGPUs) that carry out the device's functions. In various embodiments, the digital processing device further comprises an operating system configured to perform executable instructions. In various embodiments, the digital processing device can be optionally connected a computer network. In various embodiments, the digital processing device can be optionally connected to the Internet such that it accesses the World Wide Web. In various embodiments, the digital processing device can be optionally connected to a cloud computing infrastructure. In various embodiments, the digital processing device can be optionally connected to an intranet. In various embodiments, the digital processing device can be optionally connected to a data storage device.

In accordance with various embodiments, suitable digital processing devices can include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. Those of ordinary skill in the art will recognize that many smartphones are suitable for use in the system described herein. Those of ordinary skill in the art will also recognize that select televisions, video players, and digital music players with optional computer network connectivity are suitable for use in the system described herein. Suitable tablet computers include those with booklet, slate, and convertible configurations, known to those of ordinary skill in the art.

In various embodiments, the digital processing device includes an operating system configured to perform executable instructions. The operating system can be, for example, software, including programs and data, which manages the device's hardware and provides services for execution of applications. Those of ordinary skill in the art will recognize that suitable server operating systems include, by way of non-limiting examples, FreeBSD, OpenBSD, Net-BSD, Linux, Apple® Mac OS X Server®, Oracle® Solaris®, Windows Server®, and Novell® NetWare®. Those of ordinary skill in the art will recognize that suitable personal computer operating systems include, by way of non-limiting examples, Microsoft® Windows®, Apple® Mac OS X®, UNIX®, and UNIX-like operating systems such as GNU/Linux®. In various embodiments, the operating system is provided by cloud computing. Those of ordinary skill in the art will also recognize that suitable mobile smart phone operating systems include, by way of non-limiting examples, Nokia® Symbian® OS, Apple® iOS®, Research In Motion® Black-Berry OS®, Google® Android®, Microsoft® Windows Phone® OS, Microsoft® Windows Mobile® OS, Linux®, and Palm® WebOS®.

In various embodiments, the device includes a storage and/or memory device. The storage and/or memory device is one or more physical apparatuses used to store data or programs on a temporary or permanent basis. In various embodiments, the device is volatile memory and requires power to maintain stored information. In various embodiments, the device is non-volatile memory and retains stored information when the digital processing device is not powered. In various embodiments, the non-volatile memory comprises flash memory. In some embodiments, the non-volatile memory comprises dynamic random-access memory (DRAM). In various embodiments, the non-volatile memory comprises ferroelectric random-access memory (FRAM). In various embodiments, the non-volatile memory comprises phase-change random access memory (PRAM). In various embodiments, the device is a storage device including, by way of non-limiting examples, CD-ROMs, DVDs, flash memory devices, magnetic disk drives, magnetic tapes drives, optical disk drives, and cloud computing-based storage. In various embodiments, the storage and/or memory device is a combination of devices such as those disclosed herein.

In various embodiments, the digital processing device includes a display to send visual information to a user. In various embodiments, the display is a cathode ray tube (CRT). In various embodiments, the display is a liquid crystal display (LCD). In various embodiments, the display is a thin film transistor liquid crystal display (TFT-LCD). In various embodiments, the display is an organic light emitting diode (OLED) display. In various embodiments, on OLED display is a passive-matrix OLED (PMOLED) or active-matrix OLED (AMOLED) display. In various embodiments, the display is a plasma display. In various embodiments, the display is a video projector. In various embodiments, the display is a combination of devices such as those disclosed herein.

In various embodiments, the digital processing device includes an input device to receive information from a user. In various embodiments, the input device is a keyboard. In various embodiments, the input device is a pointing device including, by way of non-limiting examples, a mouse, trackball, track pad, joystick, game controller, or stylus. In various embodiments, the input device is a touch screen or a multi-touch screen. In various embodiments, the input device is a microphone to capture voice or other sound input. In various embodiments, the input device is a video camera or other sensor to capture motion or visual input. In various embodiments, the input device is a Kinect, Leap Motion, or the like. In various embodiments, the input device is a combination of devices such as those disclosed herein.

### Non-Transitory Computer Readable Storage Medium

In various embodiments, and as stated above, the systems and methods disclosed herein can include, and the methods herein can be run on, one or more non-transitory computer readable storage media encoded with a program including instructions executable by the operating system of an optionally networked digital processing device. In various embodiments, a computer readable storage medium is a tangible component of a digital processing device. In various embodiments, a computer readable storage medium is optionally removable from a digital processing device. In various embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In various embodiments, the program and instructions are permanently, substantially permanently, semi-permanently, or non-transitorily encoded on the media.

### Computer Program

In various embodiments, the systems and methods disclosed herein can include at least one computer program or use at least one computer program. A computer program includes a sequence of instructions, executable in the digital processing device's CPU, written to perform a specified task. Computer readable instructions may be implemented as program modules, such as functions, objects, Application Programming Interfaces (APis), data structures, and the like, that perform particular tasks or implement particular abstract data types. Those of ordinary skill in the art will recognize that a computer program may be written in various versions of various languages.

The functionality of the computer readable instructions may be combined or distributed as desired in various environments. In various embodiments, a computer program comprises one sequence of instructions. In various embodiments, a computer program comprises a plurality of sequences of instructions. In various embodiments, a computer program is provided from one location. In various embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In various embodiments, a computer program includes a web application. Those of ordinary skill in the art will recognize that a web application, in various embodiments, utilizes one or more software frameworks and one or more database systems. In various embodiments, a web application is created upon a software framework such as Microsoft® .NET or Ruby on Rails (RoR). In various embodiments, a web application utilizes one or more database systems including, by way of non-limiting examples, relational, non-relational, object oriented, associative, and XML database systems. In various embodiments, suitable relational database systems include, by way of non-limiting examples, Microsoft® SQL Server, mySQL™, and Oracle®. Those of ordinary skill in the art will also recognize that a web application, in various embodiments, is written in one or more versions of one or more languages. A web application may be written in one or more markup languages, presentation definition languages, client-side scripting languages, server-side coding languages, data-base query languages, or combinations thereof. In various embodiments, a web application is written to some extent in a markup language such as Hypertext Markup Language (HTML), Extensible Hypertext Markup Language (XHTML), or eXtensible Markup Language (XML). In various embodiments, a web application is written to some extent in a presentation definition language such as Cascading Style Sheets (CSS). In various embodiments, a web application is written to some extent in a client-side scripting language such as Asynchronous Javascript and XML (AJAX), Flash® Actionscript, Javascript, or Silverlight®. In various embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In various embodiments, a web application is written to some extent in a database query language such as Structured Query Language (SQL). In various embodiments, a web application integrates enterprise server products such as IBM® Lotus Domino®. In various embodiments, a web application includes a media player element. In various embodiments, a media player element utilizes one or more of many suitable multimedia technologies including, by way of non-limiting examples, Adobe® Flash®, HTML 5, Apple® QuickTime®, Microsoft® Silverlight®, Java™ and Unity®.

### Mobile Application

In various embodiments, a computer program includes a mobile application provided to a mobile digital processing device. In various embodiments, the mobile application is provided to a mobile digital processing device at the time it is manufactured. In various embodiments, the mobile application is provided to a mobile digital processing device via the computer network described herein.

A mobile application can be created by techniques known to those of ordinary skill in the art using hardware, languages, and development environments known to the art. Those of ordinary skill in the art will recognize that mobile applications can be written in several languages. Suitable programming languages include, by way of non-limiting examples, C, C++, C#, Objective-C, Java™, Javascript, Pascal, Object Pascal, Rust, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelera-tor®, Celsius, Bedrock, Flash Lite, .NET Compact Frame-work, Rhomobile, and WorkLight Mobile Platform. Other development environments are available without cost including, by way of non-limiting examples, Lazarus, Mobi-Flex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of ordinary skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nin-tendo DSi Shop.

### Standalone Application

In various embodiments, a computer program includes a standalone application, which is a program that is run as an independent computer process, not an add-on to an existing process, e.g., not a plug-in. Those of ordinary skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often per-formed, at least in part, to create an executable program. In various embodiments, a computer program includes one or more executable complied applications.

### Web Browser Plug-in

In various embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of ordinary skill in the art will be familiar with several web browser plug-ins including, Adobe® Flash® Player, Microsoft® Silver-light®, and Apple® QuickTime®. In various embodiments, the toolbar comprises one or more web browser extensions, add-ins, or add-ons. In various embodiments, the toolbar comprises one or more explorer bars, tool bands, or desk bands.

Those of ordinary skill in the art will recognize that several plug-in frame works are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

### Software Modules

In various embodiments, the systems and methods disclosed herein include a software, server and/or database modules, or incorporate use of the same in methods according to various embodiments disclosed herein. Software modules can be created by techniques known to those of ordinary skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In various embodiments, software modules are in one computer program or application. In various embodiments, software modules are in more than one computer program or application. In various embodiments, software modules are hosted on one machine. In various embodiments, software modules are hosted on more than one machine. In various embodiments, software modules are hosted on cloud computing platforms. In various embodiments, software modules are hosted on one or more machines in one location. In various embodiments, software modules are hosted on one or more machines in more than one location.

### Databases

In various embodiments, the systems and methods disclosed herein include one or more databases, or incorporate use of the same in methods according to various embodiments disclosed herein. Those of ordinary skill in the art will recognize that many databases are suitable for storage and retrieval of user, query, token, and result information. In various embodiments, suitable databases include, by way of non-limiting examples, relational databases, non-relational databases, object oriented databases, object databases, entity-relation-ship model databases, associative databases, and XML databases. Further non-limiting examples include SQL, Postgr-eSQL, MySQL, Oracle, DB2, and Sybase. In various embodiments, a database is internet-based. In further Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In various embodiments, a database is web-based. In various embodiments, a database is cloud computing-based. In other embodiments, a database is based on one or more local computer storage devices.

### Data Security

In various embodiments, the systems and methods disclosed herein include one or features to prevent unauthorized access. The security measures can, for example, secure a user's data. In various embodiments, data is encrypted. In various embodiments, access to the system requires multi-factor authentication and access control layer. In various embodiments, access to the system requires two-step authentication (e.g., web-based interface). In various embodiments, two-step authentication requires a user to input an access code sent to a user's e-mail or cell phone in addition to a username and password. In some instances, a user is locked out of an account after failing to input a proper username and password. The systems and methods disclosed herein can, in various embodiments, also include a mechanism for protecting the anonymity of users' genomes and of their searches across any genomes.

### RECITATION OF EMBODIMENTS

Embodiment 1. An interactive visualization system comprising:

a data source for obtaining a B cell receptor and/or T cell receptor data set;

a user input device for receiving a user selected parameter under which to analyze the data set;

a processor for

identifying a clonotype group in the data set using the parameter;
identifying subclonotypes within the clonotype group, wherein each identified subclonotype comprises cells having identical V(D)J transcripts, and
processing the data to define a visualization model that can display a compressed view of the identified clonotype group; and

a display for rendering a visualization of said data set according to said visualization model, wherein the visualization displays the clonotype group by identified subclonotype.

Embodiment 2. The system of Embodiment 1, wherein the parameter is a first parameter, the visualization model is a first visualization model, and the visualization is a first visualization, wherein:

the user device is further configured for receiving a second parameter under which to analyze the data set;

the processor is further configured to
re-identify a clonotype group in the data set using the second parameter;
re-identify subclonotypes within the clonotype group, wherein each identified subclonotype comprises cells having identical V(D)J transcripts; and
re-process the data to define a second visualization model that can display a modified compressed view of the identified clonotype group;

and

the display is further configured to re-render a second visualization of said data set according to said second visualization model, wherein the second visualization displays a modified version of the clonotype group by identified subclonotype.

Embodiment 3. The system of Embodiment 1, wherein the visualization displays a comparison of at least one reference sequence to a subclonotype.

Embodiment 4. The system of Embodiment 3, wherein the at least one reference sequence includes a reference sequence listing selected from the group consisting of a universal reference sequence, a donor reference sequence, and combinations thereof.

Embodiment 5. The system of Embodiment 1, wherein the visualization displays a listing of amino acid differences between each subclonotype of the clonotype population.

Embodiment 6. The system of Embodiment 1, wherein the visualization displays subclonotype information selected from the group consisting of gene expression, Hamming distance, antibody, and combinations thereof.

Embodiment 7. The system of Embodiment 6, wherein gene expression subclonotype information is selected from the group consisting of median gene expression, maximum gene expression, mean gene expression, and combinations thereof.

Embodiment 8. The system of Embodiment 7, wherein gene expression subclonotype information is reported as a UMI count.

Embodiment 9. The system of Embodiment 1, wherein for each subclonotype, the visualization displays chain-specific subclonotype information selected from the group consisting of V(D)J UMI count, V(D)J read count, constant region name, complementarity-determining region (CDR) sequence, constant sequence length, 5′UTR sequence length, differences from a universal reference constant region, differences from the 5′UTR sequence, base differences between subclonotypes, and combinations thereof.

Embodiment 10. A method for interactively visualizing and examining clonotypes within single cell datasets, the method comprising:

obtaining a B cell receptor and/or T cell receptor data set;

receiving a parameter under which to analyze the data set;

identifying a clonotype group in the data set using the parameter;

identifying subclonotypes within the clonotype group, wherein each identified subclonotype comprises cells having identical V(D)J transcripts;

processing the data to define a visualization model that can display a compressed view of the identified clonotype group;

rendering a visualization of said data set according to said visualization model, wherein the visualization displays the clonotype group by identified subclonotype.

Embodiment 11. The method of Embodiment 10, wherein the parameter is a first parameter, the visualization model is a first visualization model, and the visualization is a first visualization, the method further comprising:

receiving a second parameter under which to analyze the data set;

re-identifying a clonotype group in the data set using the second parameter;

re-identifying subclonotypes within the clonotype group, wherein each identified subclonotype comprises cells having identical V(D)J transcripts;

re-processing the data to define a second visualization model that can display a modified compressed view of the identified clonotype group; and

re-rendering a second visualization of said data set according to said second visualization model, wherein the second visualization displays a modified version of the clonotype group by identified subclonotype.

Embodiment 12. The method of Embodiment 10, wherein the visualization includes a comparison of at least one reference sequence to a subclonotype.

Embodiment 13. The method of Embodiment 12, wherein the at least one reference sequence includes a reference sequence listing selected from the group consisting of a universal reference sequence, a donor reference sequence, and combinations thereof.

Embodiment 14. The method of Embodiment 10, wherein the visualization includes a listing of amino acid differences between each subclonotype of the clonotype population.

Embodiment 15. The method of Embodiment 10, wherein the visualization includes subclonotype information selected from the group consisting of gene expression, Hamming distance, antibody, and combinations thereof.

Embodiment 16. The method of Embodiment 15, wherein gene expression subclonotype information is selected from the group consisting of median gene expression, maximum gene expression, mean gene expression, and combinations thereof.

Embodiment 17. The method of Embodiment 16, wherein gene expression subclonotype information is reported as a UMI count.

Embodiment 18. The method of Embodiment 10, wherein for each subclonotype, the visualization includes chain-specific subclonotype information selected from the group consisting of V(D)J UMI count, V(D)J read count, constant region name, complementarity-determining region (CDR) sequence, constant sequence length, 5′UTR sequence length, differences from a universal reference constant region, differences from the 5′UTR sequence, base differences between subclonotypes, and combinations thereof.

Embodiment 19. The method of Embodiment 10, further comprising receiving a user input including information configured to customize the visualization.

Embodiment 20. A graphical user interface (GUI) for displaying immune cell clonotyping information, the GUI comprising:

a listing of subclonotypes of a immune cell clonotype, wherein the subclonotypes share identical V(D)J transcripts, wherein the listing of subclonotypes includes a number of cells associated with each subclonotype;

a listing of one or more textual frames with information about chains common to each member of the immune cell clonotype, wherein the textual frame contains an amino acid sequence for the variable and constant regions of each subclonotype; and a positional information for each member of the amino acid sequence.

Embodiment 21. The GUI of Embodiment 20, wherein the listing of one or more textual frames comprises two or more textual frames.

Embodiment 22. The GUI of Embodiment 20, wherein the listing of one or more textual frames comprises two textual frames.

Embodiment 23. The GUI of Embodiment 20, wherein the listing of one or more textual frames comprises three textual frames.

Embodiment 24. The GUI of Embodiment 20, wherein the listing of one or more textual frames includes a comparison of at least one reference sequence to a subclonotype.

Embodiment 25. The GUI of Embodiment 24, wherein the at least one reference sequence includes a reference sequence listing selected from the group consisting of a universal reference sequence, a donor reference sequence, and combinations thereof.

Embodiment 26. The GUI of Embodiment 20, wherein the listing of one or more textual frames includes a listing of amino acid differences between each subclonotype of the clonotype population.

Embodiment 27. The GUI of Embodiment 20, wherein the listing of subclonotypes includes subclonotype information selected from the group consisting of gene expression, Hamming distance, antibody, and combinations thereof.

Embodiment 28. The GUI of Embodiment 27, wherein gene expression subclonotype information is selected from the group consisting of median gene expression, maximum gene expression, mean gene expression, and combinations thereof.

Embodiment 29. The GUI of Embodiment 28, wherein gene expression subclonotype information is reported as a UMI count.

Embodiment 30. The GUI of Embodiment 20, wherein for each subclonotype, the textual frame provides chain-specific subclonotype information selected from the group consisting of V(D)J UMI count, V(D)J read count, constant region name, complementarity-determining region (CDR) sequence, constant sequence length, 5′UTR sequence length, differences from a universal reference constant region, differences from the 5′UTR sequence, base differences between subclonotypes, and combinations thereof.

Embodiment 31. The GUI of Embodiment 20, further comprising a user input to receive information configured to customize the display of immune cell clonotyping information.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

1. An interactive visualization system comprising:
a data source for obtaining a B cell receptor and/or T cell receptor data set;
a user input device for receiving a user selected parameter under which to analyze the data set;
a processor for
identifying a clonotype group in the data set using the parameter;
identifying subclonotypes within the clonotype group, wherein each identified subclonotype comprises cells having identical V(D)J transcripts, and
processing the data to define a visualization model that can display a compressed view of the identified clonotype group;
and
a display for rendering a visualization of said data set according to said visualization model, wherein the visualization displays the clonotype group by identified subclonotype.

2. The system of claim 1, wherein the parameter is a first parameter, the visualization model is a first visualization model, and the visualization is a first visualization, wherein:
the user device is further configured for receiving a second parameter under which to analyze the data set;
the processor is further configured to
re-identify a clonotype group in the data set using the second parameter;
re-identify subclonotypes within the clonotype group, wherein each identified subclonotype comprises cells having identical V(D)J transcripts; and
re-process the data to define a second visualization model that can display a modified compressed view of the identified clonotype group;
and
the display is further configured to re-render a second visualization of said data set according to said second visualization model, wherein the second visualization displays a modified version of the clonotype group by identified subclonotype.

3. The system of claim 1, wherein the visualization displays a comparison of at least one reference sequence to a subclonotype, the reference sequence selected from the group consisting of a universal reference sequence, a donor reference sequence, and combinations thereof.

4. The system of claim 1, wherein the visualization displays a listing of amino acid differences between each subclonotype of the clonotype population.

5. The system of claim 1, wherein the visualization displays subclonotype information selected from the group consisting of gene expression, Hamming distance, antibody, and combinations thereof.

6. The system of claim 5, wherein gene expression subclonotype information is selected from the group consisting of median gene expression, maximum gene expression, mean gene expression, and combinations thereof.

7. The system of claim 1, wherein for each subclonotype, the visualization displays chain-specific subclonotype information selected from the group consisting of V(D)J UMI count, V(D)J read count, constant region name, complementarity-determining region (CDR) sequence, constant sequence length, 5′UTR sequence length, differences from a universal reference constant region, differences from the 5′UTR sequence, base differences between subclonotypes, and combinations thereof.

8. A method for interactively visualizing and examining clonotypes within single cell datasets, the method comprising:
obtaining a B cell receptor and/or T cell receptor data set;
receiving a parameter under which to analyze the data set;
identifying a clonotype group in the data set using the parameter;
identifying subclonotypes within the clonotype group, wherein each identified subclonotype comprises cells having identical V(D)J transcripts;
processing the data to define a visualization model that can display a compressed view of the identified clonotype group;
rendering a visualization of said data set according to said visualization model, wherein the visualization displays the clonotype group by identified subclonotype.

9. The method of claim 8, wherein the parameter is a first parameter, the visualization model is a first visualization model, and the visualization is a first visualization, the method further comprising:
receiving a second parameter under which to analyze the data set;
re-identifying a clonotype group in the data set using the second parameter;
re-identifying subclonotypes within the clonotype group, wherein each identified subclonotype comprises cells having identical V(D)J transcripts;
re-processing the data to define a second visualization model that can display a modified compressed view of the identified clonotype group; and
re-rendering a second visualization of said data set according to said second visualization model, wherein the second visualization displays a modified version of the clonotype group by identified subclonotype.

10. The method of claim 8, wherein the visualization includes a comparison of at least one reference sequence to a subclonotype, the reference sequence selected from the group consisting of a universal reference sequence, a donor reference sequence, and combinations thereof.

11. The method of claim 8, wherein the visualization includes a listing of amino acid differences between each subclonotype of the clonotype population.

12. The method of claim 8, wherein the visualization includes subclonotype information selected from the group consisting of gene expression, Hamming distance, antibody, and combinations thereof.

13. The method of claim 12, wherein gene expression subclonotype information is selected from the group consisting of median gene expression, maximum gene expression, mean gene expression, and combinations thereof.

14. The method of claim 8, wherein for each subclonotype, the visualization includes chain-specific subclonotype information selected from the group consisting of V(D)J UMI count, V(D)J read count, constant region name, complementarity-determining region (CDR) sequence, constant sequence length, 5′UTR sequence length, differences from a universal reference constant region, differences from the 5′UTR sequence, base differences between subclonotypes, and combinations thereof.

15. A graphical user interface (GUI) for displaying immune cell clonotyping information, the GUI comprising:
a listing of subclonotypes of a immune cell clonotype, wherein the subclonotypes share identical V(D)J transcripts, wherein the listing of subclonotypes includes a number of cells associated with each subclonotype;
a listing of one or more textual frames with information about chains common to each member of the immune cell clonotype, wherein the textual frame contains an amino acid sequence for the variable and constant regions of each subclonotype; and
a positional information for each member of the amino acid sequence.

16. The GUI of claim 15, wherein the listing of one or more textual frames includes a comparison of at least one reference sequence to a subclonotype, the reference sequence selected from the group consisting of a universal reference sequence, a donor reference sequence, and combinations thereof.

17. The GUI of claim 15, wherein the listing of one or more textual frames includes a listing of amino acid differences between each subclonotype of the clonotype population.

18. The GUI of claim 15, wherein the listing of subclonotypes includes subclonotype information selected from the group consisting of gene expression, Hamming distance, antibody, and combinations thereof.

19. The GUI of claim 18, wherein gene expression subclonotype information is selected from the group consisting of median gene expression, maximum gene expression, mean gene expression, and combinations thereof.

20. The GUI of claim 15, wherein for each subclonotype, the textual frame provides chain-specific subclonotype information selected from the group consisting of V(D)J UMI count, V(D)J read count, constant region name, complementarity-determining region (CDR) sequence, constant sequence length, 5′UTR sequence length, differences from a universal reference constant region, differences from the 5′UTR sequence, base differences between subclonotypes, and combinations thereof.
